(19) United States
(12) Patent Application Publication
(10) Pub. No.: US 2018/0236146 A1
(43) Pub. Date: Aug. 23, 2018

(54) VACUUM-ASSISTED FISTULA TREATMENT DEVICES, SYSTEMS, AND METHODS

(71) Applicant: CURASEAL INC., Santa Clara, CA (US)

(72) Inventors: Harold F. CARRISON, Pleasanton, CA (US); Akshay MAVANI, Los Altos, CA (US)

(21) Appl. No.: 15/795,232

(22) Filed: Oct. 26, 2017

Related U.S. Application Data
(60) Provisional application No. 62/460,850, filed on Feb. 19, 2017.

(51) Int. Cl. A61M 1/00

(52) U.S. Cl. CPC A61M 1/0084; A61B 17/0057; A61M 1/0088

(57) ABSTRACT

Devices for treating fistulas may include a cap with a vacuum port, a spacer with a lumen therethrough, a first distal anchor, and a second distal anchor. The devices may be configured to transmit negative pressure through the vacuum port, the spacer lumen, and an opening in the first distal anchor to releasably seal the first and second distal anchors to tissue, thereby securing the devices in place. The first distal anchor may also include a plurality of openings that form vacuum pathways that transmit the negative pressure. The cap may also include a flush lumen, which may allow flushing of a fistula tract without removal of the device. Methods of treating fistulas may include applying negative pressure to a fistula treatment device to anchor the device and flushing a tract of the fistula using a flushing fluid with the fistula treatment device in-situ.

### CROSS-REFERENCE TO RELATED APPLICATIONS

This application claims priority to U.S. Provisional Application Ser. No. 62/460,850 filed on Feb. 19, 2017, and titled “Vacuum-Assisted Fistula Treatment Devices, Systems, and Methods,” the content of which is hereby incorporated by reference in its entirety.

### FIELD

The present disclosure relates generally to medical devices, and more specifically, to treatment devices for fistulas.

### BACKGROUND

Fistulas are abnormal tissue-lined pathways or communications between two surfaces of the body. For example, fistulas may develop between body cavities and organs, or between cavities or organs and the surface of the body. A fistula pathway or tract includes a void in the soft tissues extending from a primary fistula opening to a blind ending or leading to one or more secondary fistula openings. Fistulas may develop due to a wound, may be the consequence of infection or abscess formation, or may be purposefully developed (e.g., tracheostomy tracts, gastric feeding tube tracts, etc.). However, most pathological fistulas are typically congenital, or result from surgery, from surgery-related complications, or from trauma. Fistulas may often have tracts or pathways that are epithelialized, endothelialized, or mucosalized.

Fistulas may form between almost any two organs. For example, fistulas may occur between internal organs and the skin (e.g., enterocutaneous fistulas, gastrocutaneous fistulas, anal fistulas, etc.), or between two internal organs (e.g., gastrointestinal fistulas, colovesicular fistulas, etc.). A perforated intestine or bowel exposed through an open abdominal wound is referred to as an “enteroatmospheric fistula.”

While some fistulas may close on their own and may not cause a person significant harm, other fistulas are chronic and may become life-threatening and may lead to death. For example, an enterocutaneous fistula between the intestinal tract and the skin may cause intestinal contents to enter into the abdomen, which can result in significant medical issues. Further, fistulas are often difficult to treat. For example, while negative pressure may often be used to treat other types of abdominal wounds, in the case of enteroatmospheric fistulas, negative pressure alone may draw enteric succus from the intestinal tract into the abdomen, which can lead to sepsis. Also, it may be difficult to simply suture or stitch an enteric fistula closed. For example, the tissue may be severely inflamed and/or damaged, and adding additional perforations to suture the tissue closed may further damage the tissue, preventing healing.

One method of treating a fistula involves surgery in which the fistula and portions of the affected organs are removed. However, this type of surgery is often a major procedure and the mortality rate may be extremely high. Furthermore, patients undergoing this type of surgery, for example, for an enterocutaneous fistula, may have chronic inflammation near the affected area, and may have dense adhesions and highly friable tissues, further complicating the procedure. Other treatment options may include implantable devices designed to aid in the closure of the fistula by the body itself. However, some of these devices may cause an adverse immunological reaction, may allow leakage of fluid from around the device, may become dislodged, or may migrate from their current position as the patient moves.

The information included in this Background section of the specification, including any references cited herein and any description or discussion thereof, is included for technical reference purposes only and is not to be regarded as subject matter by which the scope of the invention as defined in the claims is to be bound.

### BRIEF SUMMARY

Described here are devices, systems, and methods for plugging a fistula in tissue, for example, a short-tract fistula, and assisting in healing the fistula and/or controlling the flow of the fistula to allow a patient to become more medically stable. In some variations, a fistula treatment device may comprise a cap, a spacer, a first distal anchor, and a second distal anchor. The cap may be configured to be positioned on a first side of the fistula and may comprise a vacuum port. The spacer may comprise a lumen therethrough, may be coupled to the cap, and may be configured to be positioned in fistula tract. The first distal anchor may be operably connected to the cap and may be configured to be positioned on a second side of the fistula. The first distal anchor may also comprise an opening. The second distal anchor may be operably connected to the cap and the first distal anchor, and may be configured to be positioned on the second side of the fistula. The fistula treatment device may be configured to transmit negative pressure through the vacuum port, the spacer lumen, and the opening the in first distal anchor to releasably seal the first and second distal anchors to tissue on the second side of the fistula, thereby securing the device in place.

In some instances, a fistula treatment device may comprise a cap comprising a vacuum lumen, a rigid tubular spacer, a first distal anchor, and a second distal anchor. The rigid tubular spacer may be coupled to and extending from a distal surface of the cap and may comprise a spacer lumen therethrough. The spacer lumen may be fluidly coupled to the vacuum lumen. The first distal anchor may be operably connected to the cap and the tubular spacer and may comprise a plurality of openings therethrough. The plurality of openings may be fluidly coupled to the vacuum lumen through the spacer lumen. The second distal anchor may be operably connected to the cap, the tubular spacer, and the first distal anchor. The vacuum lumen, the spacer lumen, and the plurality of openings may form vacuum pathways that transmit negative pressure through the device to seal the first and second distal anchors to each other and to a tissue surface. In some variations, the cap and the tubular space may be integrally formed. In other variations, the tubular spacer may be threaded on a proximal end and/or may have a length that is adjustable. In some instances, the tubular spacer may be bio-absorbable.

In some embodiments, the vacuum lumen may extend from the spacer lumen radially outward to a proximal surface of the cap and/or the distal surface of the cap may further comprise a circular or arcuate channel formed around the tubular spacer. In some instances, the cap may further comprise a flush lumen fluidly coupling a proximal surface of the cap and the circular channel. The flush lumen may extend radially outward from the circular channel to the proximal surface of the cap. In some variations, the flush lumen and the vacuum lumen may be side-by-side, while in other variations, the vacuum lumen and the flush lumen may be vertically aligned and the vacuum lumen may be disposed proximally of the flush lumen. In some instances, a distal surface of the cap may comprise radial channels or grooves. Moreover, in some variations, at least one of the cap, the tubular spacer, the first distal anchor, and the second distal anchor may be radiopaque. In some instances, the cap, the first anchor, and the second distal anchor may be radiopaque and/or flexible.

In some embodiments, a diameter of the cap may be larger than a diameter of the second distal anchor, and the diameter of the second distal anchor may be larger than a diameter of the first distal anchor. In some instances, the openings in the plurality of openings in the first distal anchor may form a circle that is concentric with a circumference of the first distal anchor. In some variations, the plurality of openings in the first distal anchor may comprise a first set of openings and a second set of openings, each opening in the first set of openings may have a first diameter, each opening in the second set of openings may have a second diameter, and the first diameter may be smaller than the second diameter. In some embodiments, the openings in the first set of openings and the openings in the second set of openings may form concentric circles around a center point of the first distal anchor. In some instances, the first distal anchor may comprise a suture opening, and the first set of openings and the second set of openings may form concentric circles around the suture opening. In some embodiments, the first distal anchor may comprise a central suture opening. In some variations, a proximal surface of the second distal anchor may comprise one or more projections and/or the second distal anchor may comprise embedded mesh. In some embodiments, the device may further comprise a suture and the suture may be threaded through the vacuum lumen, the spacer lumen, and the suture opening, and may be coupled to the mesh. In some instances, the cap may be disc-shaped and/or at least one of the first and second distal anchors may be disc-shaped.

In some variations, the systems described here may comprise any of the above mentioned devices and a vacuum source coupled to the vacuum lumen and/or a flush source coupled to the flush lumen. For example, in some instances, the system may comprise a fistula treatment device comprising a cap comprising a vacuum lumen and optionally a flush lumen, a rigid tubular spacer, a first distal anchor, and a second distal anchor. The rigid tubular spacer may be coupled to and extending from a distal surface of the cap and may comprise a spacer lumen therethrough. The spacer lumen may be fluidly coupled to the vacuum lumen. The first distal anchor may be operably connected to the cap and the tubular spacer and may comprise a plurality of openings therethrough. The plurality of openings may be fluidly coupled to the vacuum lumen through the spacer lumen. The second distal anchor may be operably connected to the cap, the tubular spacer, and the first distal anchor. The vacuum lumen, the spacer lumen, and the plurality of openings may form vacuum pathways that transmit negative pressure through the device to seal the first and second distal anchors to each other and to a tissue surface. In some variations, the system may further comprise tubing coupling the vacuum source and the vacuum lumen, and the tubing may comprise a Y-connector with a suture seal.

In some variations, the methods described here may be methods of treating a fistula and may comprise inserting first and second distal anchors through a fistula formed between an external tissue surface and an internal tissue surface, positioning the first distal anchor and in part the second distal anchor against the internal tissue surface, positioning a cap against the external tissue surface, and sealing the first and second distal anchors to the internal tissue surface and the cap to the external tissue surface using negative pressure. In these variations, the first distal anchor may comprise a plurality of openings and the cap may comprise a flexible disc, a stiff tubular protrusion, and a continuous lumen through the flexible disc and the tubular protrusion. In some instances, the first and second distal anchors may comprise flexible discs and/or a proximal side of the second distal anchor may comprise on or more projections. In some variations, the fistula may be a short-tract fistula.

In some embodiments, positioning the cap against the external tissue surface may comprise positioning the stiff tubular protrusion within a tract of the fistula. In some variations, sealing the first and second distal anchors to the internal tissue surface and the cap to the external tissue surface may hold the fistula treatment device in place. In some of these variations, positioning the first distal anchor and in part the second distal anchor against the internal tissue surface may further comprise placing the projections of the second distal anchor in contact with a distal surface of the first distal anchor forming a gap between the first and second distal anchors. Moreover, in some of these variations, the first distal anchor may comprise a smaller diameter than the second distal anchor, and sealing the first and second distal anchors to the internal tissue surface may comprise forming a seal around a circumference of each of the first and second distal anchors. In some instances, the cap may further comprise a vacuum port fluidly coupled to the lumen, and the sealing step may further comprise applying negative pressure from the vacuum port to the gap formed between the first and second distal anchors. In some of these instances, applying negative pressure from the vacuum port to the gap may comprise applying negative pressure to the vacuum port, through the lumen, and through the plurality of openings in the first distal anchor. In some variations, applying negative pressure from the vacuum port to the gap may further comprise applying negative pressure through a fistula tract to a distal surface of the flexible disc of the cap.

In some variations, the sealing step may comprise applying negative pressure through the cap lumen, the plurality of openings in the first distal anchor, and a fistula tract external to the stiff tubular protrusion. In some of these variations, the a distal surface of the flexible disc of the cap may comprise radial channels or grooves that may transmit the negative pressure radially outward thereby increasing the surface area of the flexible disc exposed to negative pressure. In some instances, the stiff tubular protrusion may prevent collapse of the fistula when negative pressure is applied. In some embodiments, inserting the first and second distal anchors may comprise advancing a tubular delivery device carrying the first and second distal anchors therein through a fistula tract to the internal tissue surface. In some variations, the method may further comprise aligning the cap, the first distal anchor, and the second distal anchor by applying tension to a suture. The suture may be threaded through the lumen in the cap, a suture hole in the first distal anchor, and a mesh element within the second distal anchor. In some of these variations, the suture may be disposed within the lumen in the cap while sealing the first and second distal anchors to the internal tissue surface and the cap to the external tissue surface.

The methods described here may be methods of treating a fistula formed between first and second sides of tissue using a fistula treatment device. In some variations, the fistula may be a short-tract fistula. These methods may comprise applying negative pressure to the fistula treatment device to anchor the device on the first and second sides of the tissue and flushing a tract of the fistula using a flushing fluid with the fistula treatment device in-situ. In these methods, the fistula treatment device may comprise a cap, a stiff tubular spacer coupled to the cap, and first and second distal anchors operably coupled to the cap, the spacer and to one another and the cap may comprise a vacuum lumen and a flush lumen. In some variations, the cap, and the first and second distal anchors may comprise flexible discs and/or a proximal side of the second distal anchor may comprise one or more projections. In some instances, the flushing fluid may comprise saline or antibiotics. In some variations, the cap may further comprise a cellular matrix or filter.

In some instances, applying negative pressure to the fistula treatment device may comprise sealing a circumference of the cap to the first side of the tissue and sealing a circumference of each of the first distal anchor and the second distal anchor to the second side of the tissue. In some variations, the method may further comprise placing the projections of the second distal anchor in contact with a distal surface of the first distal anchor forming a gap between the first and second distal anchors. In some of these variations, the cap may further comprise a vacuum port fluidly coupled to the vacuum lumen and applying negative pressure to the fistula treatment device may comprise applying negative pressure from the vacuum port to the gap formed between the first and second distal anchors. In some of these variations, the tubular spacer may comprise a lumen therethrough and the first distal anchor may comprise a plurality of vacuum openings, and applying negative pressure from the vacuum port to the gap may comprise applying negative pressure to the vacuum port, through the vacuum lumen, the spacer lumen, and the plurality of vacuum openings in the first distal anchor. In some instances, the cap may further comprise a flush port fluidly coupled to the flush lumen, and flushing a tract of the fistula may comprise applying flushing fluid to the flush port and transporting it to the gap formed between the first and second distal anchors. In some of these instances, the first distal anchor may comprise a plurality of flush openings, and applying flushing fluid to the flush port and transporting it to the gap formed between the first and second distal anchors may comprise flowing flushing fluid through the flush lumen, down the fistula tract external to the tubular spacer, and through the plurality of flush openings.

In variations in which the tubular spacer may comprise a lumen therethrough and the first distal anchor may comprise a plurality of vacuum openings, and applying negative pressure from the vacuum port to the gap may comprise applying negative pressure to the vacuum port, through the vacuum lumen, the spacer lumen, and the plurality of vacuum openings in the first distal anchor, the cap may further comprise a fluid flush port fluidly coupled to the flush lumen, and flushing a tract of the fistula may comprise applying flushing fluid to the flush port and transporting it to the gap formed between the first and second distal anchors. In some of these variations, the first distal anchor may comprise a plurality of flush openings, and applying flushing fluid to the flush port and transporting it to the gap formed between the first and second distal anchors may comprise flowing flushing fluid through the flush lumen, down the fistula tract external to the tubular spacer, and through the plurality of flush openings.

In some of the variations described above, applying negative pressure from the vacuum port to the gap may further comprise applying negative pressure through a fistula tract to a distal surface of the cap external to the tubular spacer. In some of these variations, the distal surface of the cap may comprise radial channels or grooves that transmit the negative pressure radially outward thereby increasing the surface area of the cap exposed to negative pressure. In some of these variations, the tubular spacer may prevent the cap and the first distal anchor from contacting one another when negative pressure is applied.

In some instances, the methods of treating a fistula formed between first and second sides of tissue using a fistula treatment device may further comprise aligning the cap, the first distal anchor, and the second distal anchor by applying tension to a suture. The suture may be threaded through the vacuum lumen, a lumen in the tubular spacer, a suture hole in the first distal anchor, and mesh within the second distal anchor. In some of these instances, the suture may be disposed within the vacuum lumen while applying negative pressure to the fistula treatment device.

In some variations, flushing the fistula tract of the fistula may comprise flushing tissue surfaces under the cap, under the first distal anchor, and under the second distal anchor. In some of these variations, flushing a tract of the fistula may further comprise removing a portion of the flushing fluid through a lumen in the tubular member and the vacuum lumen.

### BRIEF DESCRIPTION OF THE DRAWINGS

Various objects and advantages and a more complete understanding of the present invention are apparent and more readily appreciated by reference to the following Detailed Description and to the appended claims when taken in conjunction with the accompanying Drawing wherein:

FIG. 1A depicts a side view of a variation of fistula treatment device and FIG. 1B depicts a schematic cross-sectional view of the variation shown in FIG. 1A.

FIGS. 2A-2E depict a variation of a cap comprising vertically aligned lumens. FIG. 2A is an angled top view, FIGS. 2B and 2D are bottom views, FIG. 2C is a cross-sectional view taken along line A-A in FIG. 2B, and FIG. 2E is a perspective cross-sectional view.

FIGS. 3A-3E depict a variation of a cap comprising laterally aligned lumens. FIG. 3A is an angled top view, FIGS. 3B and 3D are bottom views, FIG. 3C is a cross-sectional view taken along line B-B in FIG. 3B, and FIG. 3E is a perspective cross-sectional view.

FIGS. 4A and 4C are top views of a variation of a first distal anchor and FIGS. 4B and 4D are cross-sectional views taken along line C-C in FIG. 4A and line D-D in FIG. 4C, respectively.

FIG. 5A is a top view of a variation of a second distal anchor and FIG. 5B is a cross-sectional view taken along line E-E in FIG. 5A.

FIGS. 6A and 6B depict an angled top view and a cross-sectional view, respectively, of a distal portion of a variation of fistula treatment device in a concentrically aligned configuration.

FIGS. 7A-7E depict a variation of a fistula treatment device. FIG. 7A depicts a cross-sectional view of a variation of a fistula treatment device, FIG. 7B depicts a schematic cross-sectional view of the fistula treatment device depicted in FIG. 7A implanted in tissue, and FIG. 7C depicts the fistula treatment device depicted in FIG. 7A implanted in tissue and coupled to vacuum and flushing tubing. FIGS. 7D and 7E illustrate variations of the vacuum and fluid flow paths of the fistula treatment device, respectively.

FIGS. 8A and 8B depict a bottom view and cross-sectional view along line A-A, respectively, of a variation of a cap with integrated radial channels.

FIGS. 9A and 9B depict a bottom view and a cross-sectional view along line B-B, respectively, of another variation of a cap with integrated radial channels.

FIG. 10 depicts a variation of a distal anchor comprising a plurality of anchor portions.

FIGS. 11A-11D depict a top perspective view, a top view, a bottom perspective view, and a bottom view, respectively, of a variation of an anchor comprising a curved shape.

### DETAILED DESCRIPTION

Embodiments of a fistula treatment device for treating enteroatmospheric fistulas, short-tract fistulas, and other openings in tissue, such as wounds, are described herein. In some embodiments, at least a portion of the device may cover and seal a fistula or wound opening to promote healing or help stabilize a patient's health. The device may be sealed to tissue using negative pressure and may be held in place without requiring sutures or stitching. In some variations, the device may also be used to flush or clean the wound while the device remains implanted. The device may be used for a number of different types of fistulas, tissue openings, or other wounds.

The device may include a cap and first and second distal anchors, and the cap and distal anchors may be connected together via a spacer or tubular element comprising a continuous, enclosed lumen therethrough. In some variations, the device may include a single distal anchor (e.g., only the first distal anchor). The enclosed lumen may transmit negative pressure applied at the cap to the distal anchors, which may pull the cap and the distal anchors toward the spacer, seal the cap and distal anchors to tissue surfaces thus sealing the tissue opening. In some variations, the spacer or tubular element is omitted.

The cap of the device may be configured to be positioned on a first or exterior surface of the damaged tissue, or against or over a first (e.g., proximal) opening of the fistula (e.g., a short-tract fistula). In some instances, a fistula may have more than one opening on a first side (external side) of the fistula. In these variations, the cap may be positioned to cover all of the fistula openings on the first side of the fistula. The cap may, for example, be made of one or more impermeable biocompatible and/or bioabsorbable materials. As such, the cap may be capable of preventing liquids (e.g., enteric succus) and other materials from entering into or exiting out of the fistula opening, while also substantially preventing damage to, and an autoimmune response from, the body as the material may be eventually absorbed by the body. This is important, for example, in the case of an intestinal fistula, where the impermeable cap of the device may prevent, resist or reduce intestinal matter and liquids from passing through the fistula into or onto the abdominal cavity. Similarly, the distal anchor(s) may be configured to be positioned over a second (e.g., distal) opening of the fistula (e.g., an opening in an interior surface of the damaged tissue) and may, for example, be made of one or more impermeable biocompatible and bioabsorbable materials. Other appropriate materials may alternatively or additionally be used. The cap and distal anchor(s) may be operably connected to one another, sandwiching the tissue surrounding the fistula therebetween. The cap and the distal anchor(s) may be connected together via a suture that may be threaded through the lumen in the spacer, and the distal anchor(s). The suture may be any suitable material and may be resorbable or non-resorbable. In some variations, the ends of the suture may be attached to the skin of a patient, or a fitting (e.g., a Tuohy borst fitting) may be used to hold the suture ends, which may allow the distal anchor(s) to be loosened.

The distal anchor(s) may be configured to be inserted into the fistula opening and then expanded and manipulated. This expansion and manipulation may position the distal anchor(s) on the second or interior side of the tissue. The device may comprise first and second distal anchors of varying diameter that may be folded or compressed and placed within a tubular delivery device, which may be inserted into the first opening of the fistula and advanced to a second opening of the fistula. The first and second distal anchors may then be deployed from the tubular delivery device such that the distal anchors expand or unfurl on a second side of the fistula adjacent to the second opening of the fistula. In some instances, a fistula may have more than one opening on a second side (internal side) of the fistula. In these variations, the distal anchor(s) may be positioned to cover all of the fistula openings on the second side of the fistula. In variations in which a single distal anchor is used, the single distal anchor may be similarly delivered.

The fistula treatment devices described here may comprise vacuum pathways that may transmit negative pressure through the fistula treatment device to seal the single distal anchor to a tissue surface or seal the first and second distal anchors to each other and to a tissue surface. For example, in some variations, a vacuum pathway may be formed through a vacuum port and vacuum lumen in the cap, through a lumen in the spacer, and through an opening in the first distal anchor. In some instances, a plurality of vacuum pathways may be formed, each utilizing the vacuum port, vacuum lumen, and spacer lumen, but each comprising its own opening in the distal anchor. In variations comprising two distal anchors, the vacuum pathway may connect the vacuum port on a proximal or outer surface of the cap, with a gap that is formed between the distal anchors when deployed. In variations comprising a single or multiple distal anchors, the cap and first distal anchor may comprise spreading or distribution features (e.g., radial grooves or channels) that may assist in transmitting negative pressure to the edges of the cap, the first distal anchor, and in some variations the second distal anchor, thereby enhancing the ability of the device to seal to tissue.

In some variations, the fistula treatment device may also comprise a fluid flushing pathway that may enable a user to flush or otherwise clean a wound without removing the fistula treatment device. For example, the flushing fluid pathway may be formed through a flush port, flush lumen, and a circular or semi-circular groove in the cap, and through an opening in the distal anchor. In some variations, a plurality of flushing fluid pathways may also be formed, each utilizing the flush port, flush lumen and circular or semi-circular groove, but each comprising its own opening in the distal anchor. In some variations, the spreading or distribution features (e.g., radial grooves or channels) that may assist in transmitting negative pressure to the edges of the device, may also assist in spreading flushing fluid.

Referring now to the drawings, where like or similar elements are designated with identical reference numerals throughout the several views. Those skilled in the art can readily recognize that numerous variations and substitutions may be made in the invention, its use and its configuration to achieve substantially the same results as achieved by the embodiments described in the specification.

### Devices

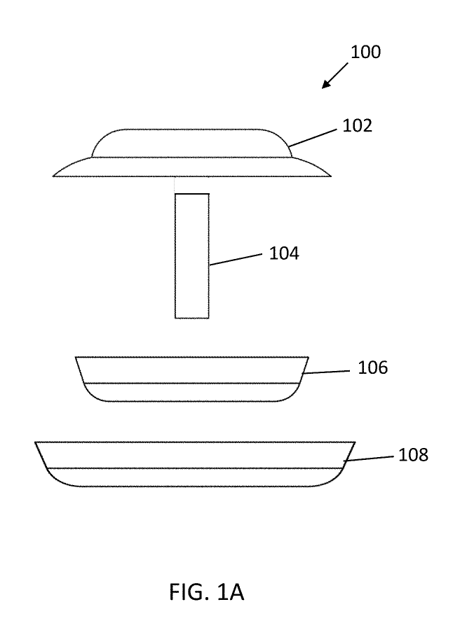

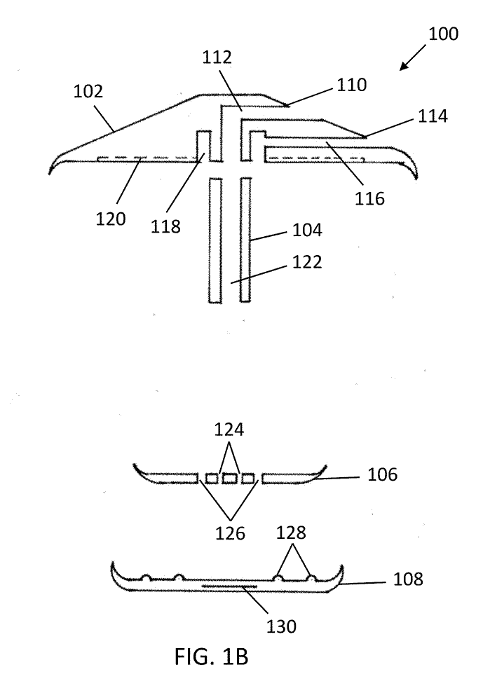

FIG. 1A depicts a side-view of a variation of the fistula treatment devices described here. As shown there, the fistula treatment device (100) may comprise a cap (102), a spacer (104), a first distal anchor (106), and a second distal anchor (108). The cap (102), the spacer (104), the first distal anchor (106), and the second distal anchor (108) may be operably connected to one another using a suture (not shown). When operably coupled together, the spacer (104) may be disposed between the cap (102) and the first distal anchor (106), and the first distal anchor may be disposed between the spacer (104) and the second distal anchor (108). The suture may be useful in not only coupling the cap (102), the spacer (104), and the distal anchors (106, 108) together, but also in appropriately aligning the elements. For example, the suture may be used to position the spacer (104) at or near the center of the cap (102) and the distal anchors (106, 108), and concentrically align the first and second distal anchors (106,108). Although first and second distal anchors (106, 108) are shown, in some variations, the fistula treatment device (100) may only comprise a first distal anchor (106).

While depicted as separate elements, in some variations the cap (102) and the spacer (104) may be integrally formed or interconnected in some fashion. In other variations, the spacer (104) may be coupled to the cap (102) using any suitable technique. For example, in some instances, the proximal end of the spacer (104) may be threaded and may be screwed into a threaded opening in a distal surface of the cap (102) and/or the spacer (104) may be coupled to the cap (102) using adhesive. The spacer may be resorbable or non-resorbable.

In some variations, the size of the spacer may be customizable. For example, the length of the spacer and/or the diameter of the spacer may be adjustable. In some variations, the length of the spacer may be modified by cutting a distal portion of the spacer after determining (e.g., through measurement of the length of the fistula tract) the spacer length needed. In other variations, the length of the spacer may be adjustable without permanently removing a portion of the spacer (e.g., by screwing or unscrewing the spacer such that it is advanced from or retracted into the cap). In variations in which the spacer diameter may be adjustable, the spacer may itself be inflatable and/or the device may comprise a bushing coupled to the spacer. The bushing may comprise an inflatable or expandable sheath or tube that circumferentially surrounds the spacer such that, when inflated or expanded, the spacer and bushing have a larger diameter than the spacer alone. An inflatable spacer and/or inflatable bushing may be expanded using any suitable means, for example, air or fluid (e.g., saline), to increase the space occupied by the spacer. In some instances, an inflatable spacer or bushing may be expanded after placement within a fistula tract (i.e., in-situ within the fistula tract) using an inflation line. In other variations, the inflatable spacer or bushing may be expanded prior to placement within the fistula tract, inserted within the fistula tract to confirm that the diameter is appropriate for the particular patient, and if not, it may be removed and further expanded or deflated to increase or decrease the diameter. In other variations, the spacer may be formed from or may comprise wrapped or rolled filaments or sheets that may be unrolled to customize the diameter of the spacer for a particular patient. In some instances, the spacer may be provided with a maximum diameter, and the filaments or sheets may be unrolled or otherwise removed to decrease the diameter of the spacer. In some of these variations, the spacer may comprise a core and the filaments or sheets may be wrapped or rolled around the core. In other variations, the filaments and/or sheets may be rolled upon themselves to form the spacer.

In other instances, the device may comprise multiple spacers of varying lengths and/or diameters, and the spacer with the appropriate length and/or diameter may be selected based on the particular patient's needs. In these variations, a first spacer may be selected at the beginning of treatment, and the first spacer may be removed and replaced with a spacer having one or more different dimensions (e.g., with a smaller diameter) after an appropriate length of time. This may allow a fistula tract diameter to become smaller with each smaller spacer that is used.

The cap (102) and distal anchors (106, 108) may have any shape suitable for covering a fistula opening, for example, they may be circular, square, rectangular, oval, triangular, a combination thereof, or the like. In some variations, the cap (102), and the first and second distal anchors (106, 108) may be disc-shaped. In some variations, one or more of the cap (102), and the first and second distal anchors (106, 108) may comprise a single sealing ring or a plurality (e.g., two, three, or more) of sealing rings.

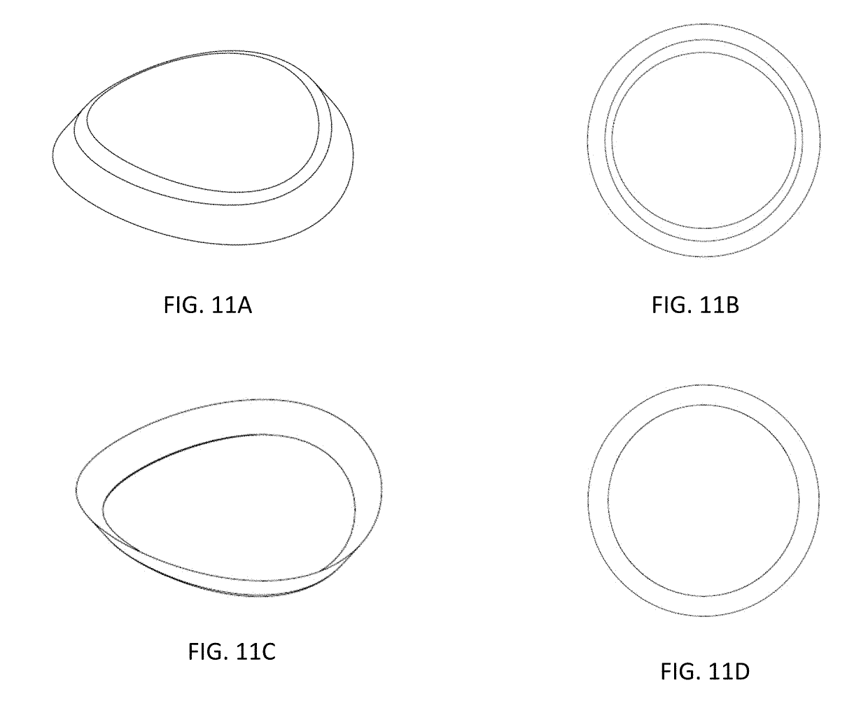

In some variations, the cap, the first distal anchor, and/or the second distal anchor may be curved to fit the diameter of the fistula opening. For example, the cap, the first distal anchor, and/or the second distal anchor may comprise a hyperbolic paraboloid or saddle shape. For instance, as depicted in FIGS. 11A-11D, the cap, first distal anchor, and/or second distal anchor may have a first curvature on a top or first surface along a first axis, and a second curvature on a second, opposite or bottom surface along a second axis that is orthogonal to the first axis. For example, in some variations, the first curvature may have a radius of curvature of between about 1.26 inches and about 1.76 inches, about 1.30 inches and about 1.70 inches, about 1.35 inches and about 1.65 inches, or about 1.45 inches and about 1.55 inches, and/or the second curvature may have a radius of curvature of between about 1.33 inches and about 1.83 inches, about 1.45 inches and about 1.75 inches, about 1.50 inches and about 1.70 inches, or about 1.55 inches and about 1.65 inches. In some variations, the first curvature may have a radius of curvature of about 1.51 inches and the second curvature may have a radius of curvature of about 1.58 inches. Additionally or alternatively, the cap, first distal anchor, and/or second distal anchor may have rounded edges between the top surface and a side or circumferential surface, and/or the side or circumferential surface (sidewall) may be angled or sloped outward (i.e., away from a center point). For example, in some variations, the angle formed between the first or top surface and the side or circumferential surface may be between about 25 degrees and about 85 degrees, about 30 degrees and about 70 degrees, about 35 degrees and about 65 degrees, about 45 degrees and about 60 degrees, or about 50 degrees and about 55 degrees. In some variations, the angle may be about 55 degrees. Additionally or alternatively, the side or circumferential surface may have a height between about 0.20 inches and about 0.40 inches, about 0.25 inches and about 0.35 inches, about 0.25 inches and about 0.30 inches, about 0.15 inches and about 0.25 inches, about 0.18 inches and about 0.23 inches, about 0.20 inches and about 0.22 inches. In some instances, the cap, first distal anchor, and/or second distal anchor may have a generally frustoconical shape.

The spacer (104) may have any shape suitable for placement within a fistula tract, for example, the spacer (104) may be a cylinder, a rectangular prism, a triangular prism, a cone, a hexagonal prism, or the like, and may have a continuous, fully enclosed, straight lumen therethrough (i.e., from a proximal to a distal end of the spacer (104)). The spacer lumen may be parallel to or disposed along a longitudinal axis of the spacer (104) and may be used for a variety of purposes, for example, to transmit negative pressure (i.e., as a vacuum lumen), to transmit flushing fluid (i.e., as a flush lumen), or to transmit both (i.e., as a combination vacuum and flush lumen). In some instances, the spacer may comprise a plurality of lumens (e.g., two, three, four, or more), which may be used for the same or different purposes. For example, in some variations, the spacer may comprise a first lumen, which may serve as a flush lumen, and a second lumen, which may serve as a vacuum lumen. In variations comprising multiple lumens, the cap may comprise a single port in fluid communication with each lumen or the cap may comprise a plurality of ports, and each port may be in fluid communication with a different lumen. In some variations, the spacer may comprise openings between an external surface of the spacer and the lumen, which may assist in transmitting negative pressure and/or flushing fluid from the lumen of the spacer to the fistula tract. Exemplary lengths for the spacer may include from about 0.075 inches (0.191 cm) to about 0.625 inches (1.588 cm) and from about 0.05 inches (0.127 cm) to about 5.00 inches (12.70 cm). In some variations, the spacer may have a length between about 0.125 inches (0.318 cm) and about 5.00 inches (12.70 cm), between about 0.150 inches (0.381 cm) and about 0.300 inches (0.762 cm), between about 0.200 inches (0.508 cm) and about 0.270 inches (0.686 cm), or may be about 0.250 inches (0.635 cm). The diameter or maximum transverse dimension of the spacer (constant or adjustable diameter/maximum transverse dimension) may be between about 0.05 inches (0.127 cm) and about 1.50 inches (3.81 cm). In some variations, the diameter or maximum transverse dimension of the spacer may be between about 0.250 inches (0.635 cm) and about 0.500 inches (1.27 cm), between about 0.270 inches (0.686 cm) and about 0.350 inches (0.889 cm), or may be about 0.3125 inches (0.794 cm).

The cap (102), the first distal anchor (106), and/or the second distal anchor (108) may be flexible, and may be formed of one or more fluid-impermeable materials (e.g., fluid-impermeable silicon), which may generally prevent or at least partially block fluids and other materials from passing around and through the cap, the first distal anchor, and/or the second distal anchor. The spacer (104) may also be formed of one or more fluid-impermeable materials (e.g., fluid-impermeable silicon), however, the spacer may be rigid or stiff such that it does not buckle or fold when compressive force is applied to its ends. Put another way, the spacer may have enough column strength to resist longitudinal compression when negative pressure is applied, but may still flex off-axis. Additionally, the spacer may still be at least partially axially compliant such that it may absorb length changes of the fistula as a patient moves. In some variations, the rigidity of the spacer may be attributable to its thickness. For example, the spacer (104) may be substantially thicker than the cap, the first distal anchor, and/or the second distal anchor, which may result in the spacer being less flexible than the distal anchors. Additionally, in some variations, the cap (102), the spacer (104), and the first and second distal anchors (106, 108) may be bioabsorbable (e.g., may be formed from bioabsorbable polymers), so that one or more parts of the device may eventually be absorbed by a patient's body and/or radiopaque, so that one or more parts of the device may be visualized using x-ray. In some variations, one or more of the cap (102), the spacer (104), the first distal anchor (106), and the second distal anchor (108) may comprise a radiopaque marker (e.g., a ring). In some variations, one or more of the cap (102), the spacer (104), the first distal anchor (106), and the second distal anchor (108) may be formed from or may have features (e.g., seals) formed from radiopaque doped material, for example, silicone doped with barium sulfate or tungsten. Moreover, in some variations, the spacer (104) may include tissue growth enhancing material (e.g., collagen) which may allow tissue to grow into and/or around the tissue growth enhancing material.

In some variations, the cap, the spacer, the first distal anchor, and/or the second distal anchor may comprise one or more of the following: PLA (polylactic acid), PGA (polyglycolic acid), P4HB (poly-4-hydroxybutrate), PCL (polycaprolactone), PLGA (poly lactide-co-glycolide), and PLLA (poly-L-lactide).

The fistula treatment device may be at least partially implantable within the body in order to cover and seal the openings of a fistula. The cap (102) may be configured to be positioned on a first side of a fistula and may be sized and shaped to cover a first opening of the fistula. For example, in variations in which the cap is disc-shaped, the diameter of the cap may be larger than the diameter of the first opening of the fistula. The spacer (104) may be configured to be positioned in the fistula tract, and may both prevent the fistula from collapsing and prevent the cap and the distal anchors from being pulled into contact with one another when negative pressure is applied to hold the device in place. The first and second distal anchors (106, 108) may be configured to be positioned on a second side of the fistula, and may each be sized and shaped to cover the second opening of the fistula. The first distal anchor (106) may be positioned between the second distal anchor (108) and the second opening of the fistula, however, the first distal anchor (106) may be smaller (e.g., have a smaller diameter) than the second distal anchor (108) such that the second distal anchor (108) may still contact tissue around the second opening of the fistula. Utilizing a larger second distal anchor (108) (e.g., a larger diameter, circumference, or the like) may be useful in that a seal may be formed not only between the first distal anchor (106) and the tissue surface surrounding the second opening, but also between the second distal anchor (108) and the tissue adjacent to and around the first distal anchor (106). This dual-seal may result in a stronger anchor point for the device.

As mentioned above, the fistula treatment devices described here may be held in place in the body using negative pressure and in some variations, may also be used to assist in flushing or debriding a wound. The device may comprise features that may assist in transmission of negative pressure through the device and optionally application of flushing fluid to the wound (e.g., tissue around the fistula and the fistula tract). FIG. 1B depicts a schematic cross-sectional view of the fistula treatment device depicted in FIG. 1A in which some of these additional features can be seen.

For example, the cap (102) may comprise a vacuum port (110) coupled to or integrally formed with a vacuum lumen (112), a flush port (114) coupled to or integrally formed with a flush lumen (116), a circular or semi-circular channel, groove, or trough (118) in fluid communication with the flush lumen (116), and one or more radial channels or grooves (120). The arcuate or circular groove (118) may be formed in a distal surface of the cap (102) and may allow the flushing fluid to flow along most if not all of the distal surface of the cap (102), (e.g., the underside of the cap (102), which contacts the tissue in which the fistula is formed) thereby enabling a larger portion of the tissue surface under the cap to be exposed to flushing fluid, and flushing of the wound without removal of the cap. The radial channels (120) may assist in distributing negative pressure radially outward (i.e., from the center of the cap toward the outer edges), and in carrying the flushing fluid radially outward, as will be discussed in more detail below. In some variations, the vacuum port (110) and the flush port (114) may simply be the proximal opening of the vacuum lumen (112) and the flush lumen (116) respectively, while in other variations, the vacuum port (110) and/or flush port (114) may comprise additional elements configured to connect to a vacuum and/or flushing fluid source respectively, such as tubing connectors.

Additionally, the spacer (104) may comprise a central spacer lumen (122), which may be fluidly coupled to the vacuum port (110) and the vacuum lumen (112). The spacer lumen (122) may transmit negative pressure from the vacuum lumen (112) to the distal anchors (106, 108), and in some variations, may allow contaminated flushing fluid to be removed from the body. The first distal anchor (106) may comprise one or more vacuum openings (124) that may transmit negative pressure through the first distal anchor (106) to the second distal anchor (108). The first distal anchor (106) may also comprise one or more flushing fluid openings (126) that may allow flushing fluid to pass through the first distal anchor (106) to the second distal anchor (108), which may facilitate cleaning between the first and second distal anchors (106, 108) and between the second distal anchor (108) and tissue. Finally, the second distal anchor (108) may comprise one or more (e.g., a plurality, two, three, four, five, ten, twelve, sixteen, eighteen, or more) projections (128) formed on a proximal side or surface thereof. The projections (128) may serve as stand-offs such that they form a gap between the first and second distal anchors (106, 108) when the anchors are placed in contact with one another (e.g., stacked), which allows the negative pressure and the flushing fluid to act on a larger surface area of the second distal anchor (108) and enables negative pressure and flushing fluid to reach an outer edge (e.g., circumference) of the second distal anchor (108). In some variations, the first distal anchor (106) may comprise one or more projections formed on a distal side or surface thereof instead of, or in addition to, the projections formed on the proximal side or surface of the second distal anchor (108).

Also depicted in FIG. 1B is a mesh element (130) (e.g., a mesh sheet) embedded within the second distal anchor (108). As mentioned above, a suture may operable connect the cap (102), the spacer (104), the first distal anchor (106), and the second distal anchor (108). The mesh element (130) may prevent the suture from being torn out or otherwise removed from the second distal anchor when a tensioning or other force is applied to a proximal end of the suture. Although not depicted in FIG. 1B, the suture may be disposed within the vacuum lumen (112) and the spacer lumen (122), and may be threaded through a center point in the first distal anchor (106) and the mesh element (130) in the second distal anchor (108). Threading the suture through the first distal anchor (106) may form a central suture opening in the first distal anchor (106). The suture may be used to align the elements with respect to one another and may provide a back-up or alternative method of anchoring the fistula treatment device should the negative pressure fail or be undesirable for any reason.

As mentioned above, the cap (102) may be sized and shaped to cover and seal a first opening of a fistula, and the first and second distal anchors (106, 108) may be sized and shaped to cover and seal a second opening of a fistula. In variations in which the cap and the distal anchors are disc-shaped, the diameter of the cap may be larger than the diameters of both the first and second distal anchors. Additionally, the diameter of the second distal anchor may be great than the diameter of the first distal anchor or vice versa. Using a large (relative to the distal anchors) cap may provide a better holding force when negative pressure is applied as a larger diameter disc has a larger surface area with which it may attach to tissue. Moreover, as discussed briefly above, utilizing a second distal anchor with a larger diameter than the first distal anchor allows both the first and second distal anchors to contact and form a seal with tissue. Additionally, utilizing a second distal anchor with a larger diameter than the first distal anchor may result in a ring of tissue located between the outer edges of the first and second distal anchors to be pulled toward the second distal anchor, forming a lip or raised tissue surface that may assist in holding the device in place.

Exemplary diameters or maximum transverse dimensions (e.g., relative to a longitudinal axis of the spacer or a longitudinal axis of the fistula) for the cap, the first distal anchor, and the second distal anchor are about 2.50 inches (6.35 cm), about 0.875 inches (2.22 cm), and about 1.375 inches (3.49 cm), respectively. In some variations, the diameter or maximum transverse dimension of the first distal anchor and/or the second distal anchor may be between about 0.25 inches (0.635 cm) and about 2.00 inches (5.08 cm). In some instances, the difference between the diameters of the first and second distal anchors may be between about 0.10 inches (0.254 cm) and 0.60 inches (1.524 cm). As used herein, “about” means ±5%.

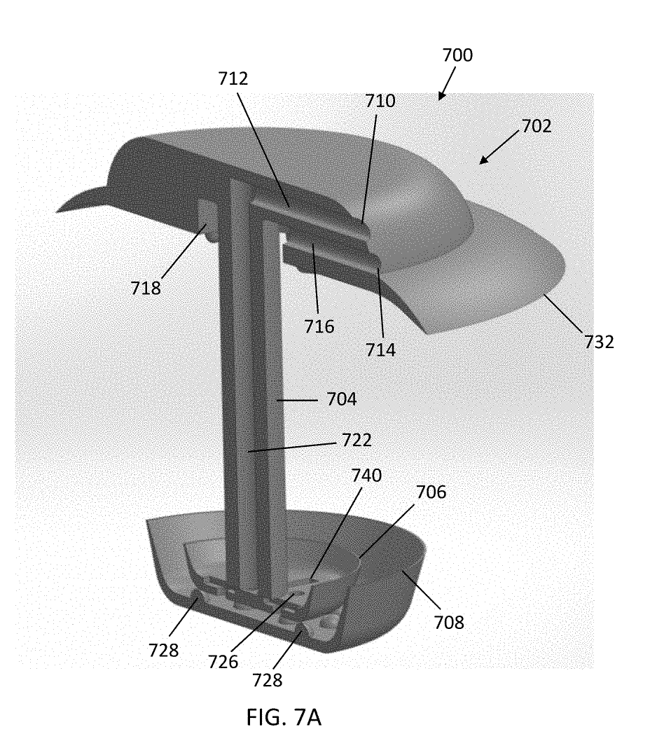

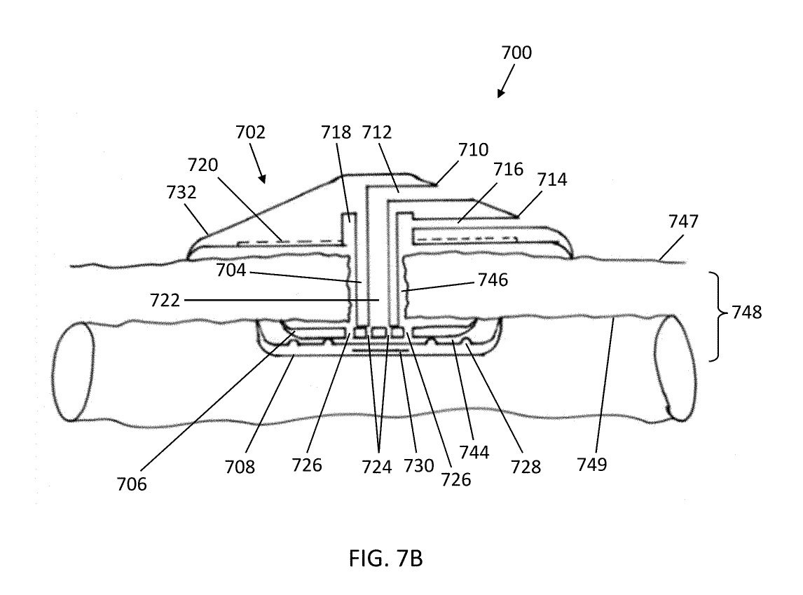

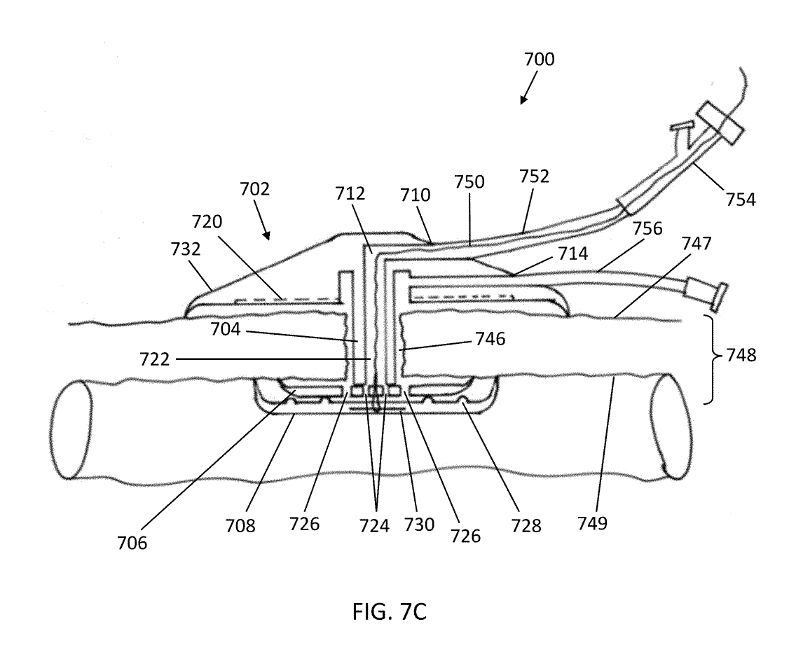

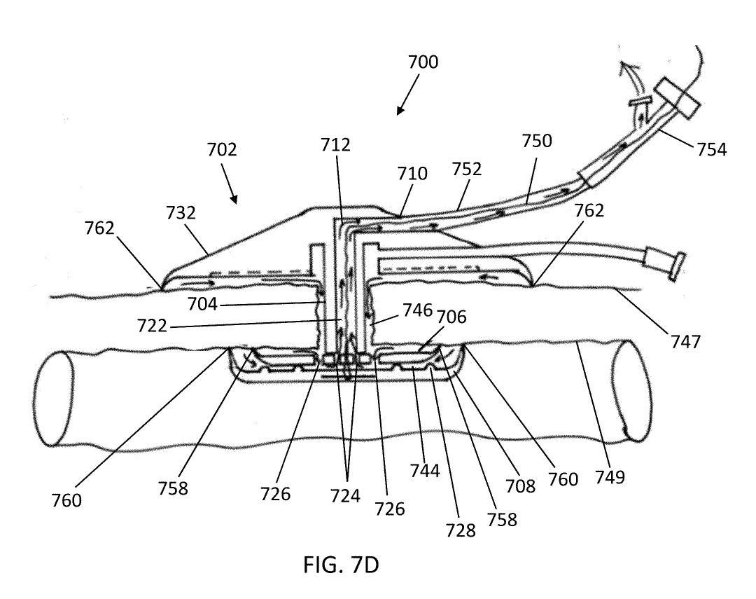

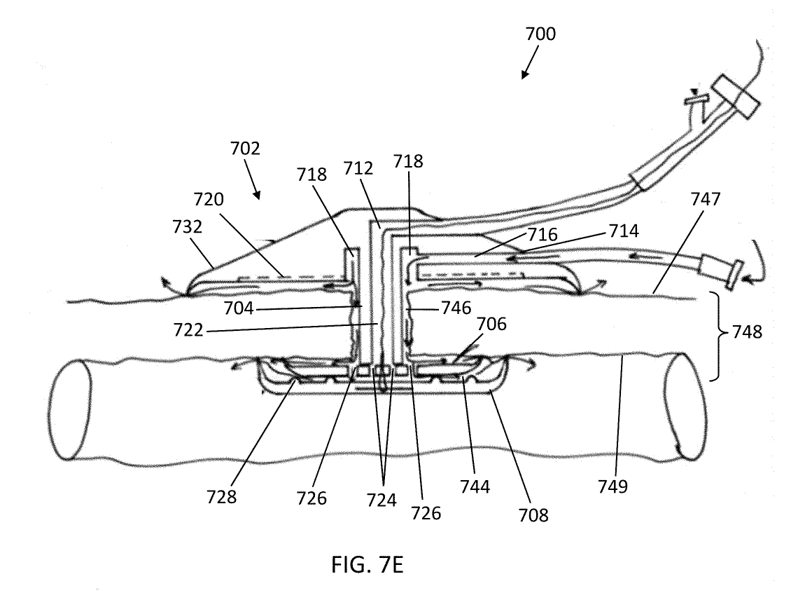

Moreover, as discussed above, the cap, and the first and second distal anchors may each be disc-shaped in that they may comprise flat central portions with curved outer edges, shown for the distal anchors depicted in FIG. 1B. In some variations, the cap and the first and second distal anchors may be dome-shaped. In some instances, the cap may comprise a first portion with a first diameter and a first radius of curvature and a second portion with a second diameter and a second radius of curvature, for example, as shown in FIG. 7A. In some of these variations, the diameter of the first portion may be smaller than the diameter of the second portion, but the first radius of curvature may be greater than the second radius of curvature. Additionally, the first portion may be stacked on top of or may otherwise be proximal to the second portion such that the cap is thicker in the center than along the outer edges (circumference).

In some variations, the device may comprise a feature designed to prevent the vacuum lumen from becoming clogged with debris. For example, in some embodiments, the cap may comprise a cellular matrix (e.g., an open cell foam, sponge) or other filter coupled to the distal surface thereof (on the tissue contacting surface), and/or may comprise a plurality of entrance/exit points in the vacuum lumen. In other variations, the first distal anchor may comprise a cellular matrix or filter coupled to a proximal surface thereof (on the tissue contacting surface), and/or may comprise a plurality of entrance/exit points for the negative pressure. In some variations, the cap and the first distal anchor may both comprise a cellular matrix or filter. For example, in some instances, the cap may comprise a cellular matrix and the first distal anchor may comprise a filter, or vice versa. In some instances, the cellular matrix or filter may have any suitable cross-sectional shape, for example, circular, square, oval, triangular, hexagonal, octagonal, or the like, the maximum transverse dimension (e.g., length or width) or diameter may be about 0.835 inches (21.21 mm), and the thickness may be between about 0.077 inches (1.96 mm) and about 0.308 inches (7.82 mm). In some variations, the cellular matrix or filter may be between about 0.154 inches (3.91 mm) and about 0.308 inches (7.82 mm) thick.

### Cap

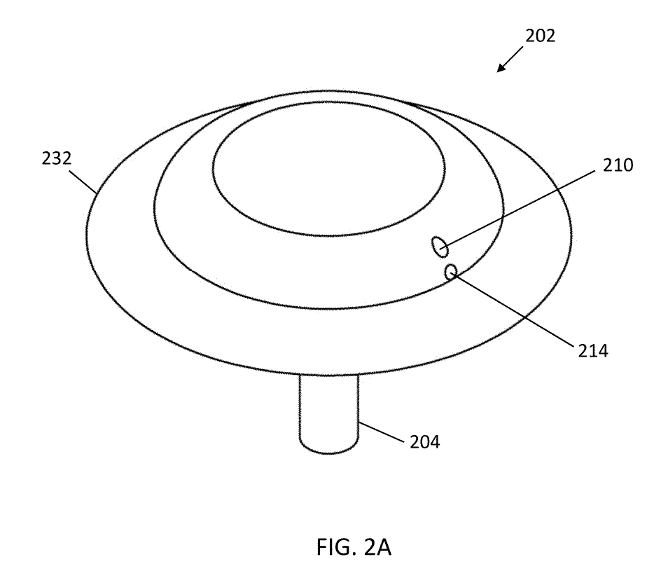

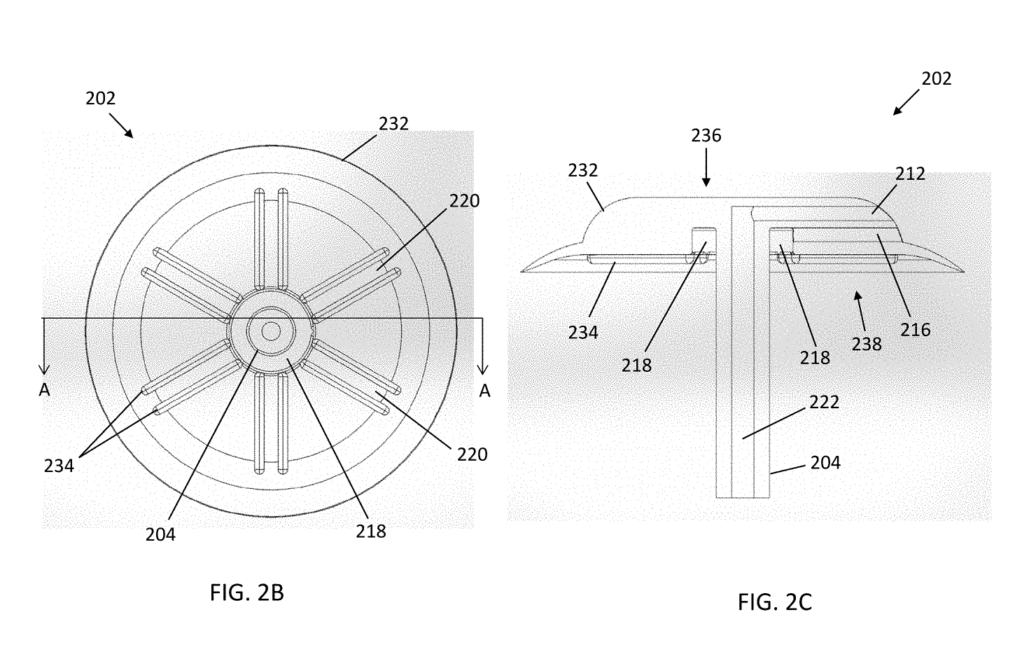

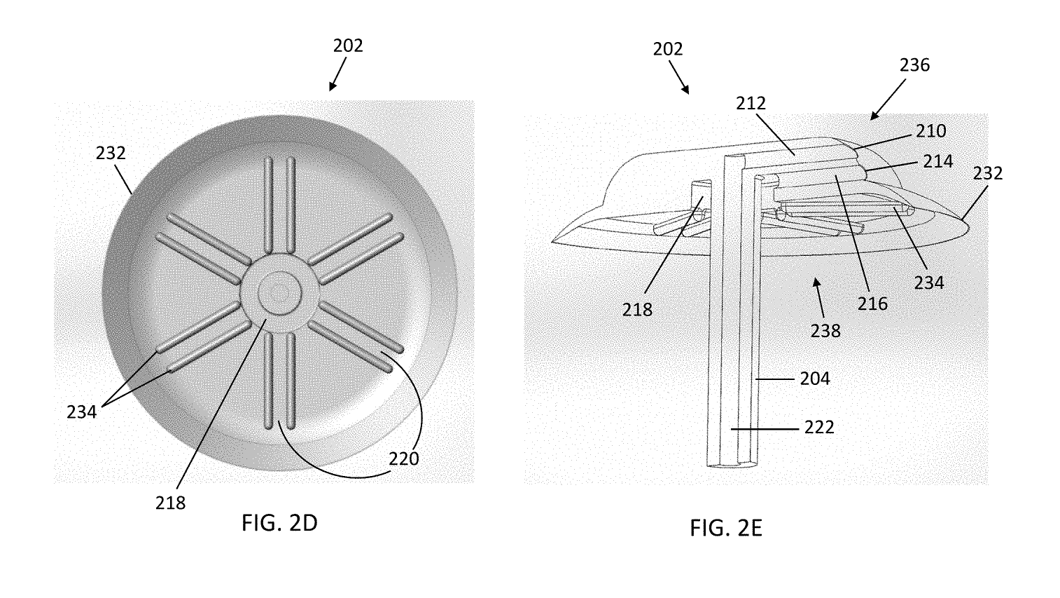

FIGS. 2A-2E illustrate a variation of a cap (202) of a fistula treatment device with vertically aligned vacuum and flush lumens. In the variation shown, the cap (202) comprises a flexible disc (232) and an elongate, rigid, tubular spacer (204) comprising a lumen therethrough (222). The tubular spacer (204) may be coupled to (including formed integrally with) and extending distally from the flexible disc (232). The cap (202) may further comprise a plurality of ports, for example, a vacuum port (210) and a flush port (214) fluidly coupled to a vacuum lumen (212) and a flush lumen (216), respectively. The vacuum lumen (212) and the flush lumen (216) may be substantially vertically aligned such that the vacuum lumen (212) may be located proximally of the flush lumen (216). The cap (202) may further comprise a circular channel or groove (218) partially or fully circumferentially surrounding an external surface of the tubular spacer (204), which may assist in flushing fluid distribution. The cap (202) may further comprise one or more radial channels (220), which may to assist negative pressure and optionally flushing fluid distribution.

In some variations, the cap (202) may further comprise one or more sealing rings formed on or from a distal surface of the flexible disc (232). The sealing rings may be full rings, interrupted rings, portions of rings, or form any other shape that provides a sealing or healing benefit. In some variations, the cap (202) may comprise two concentric sealing rings. In some instances, the sealing rings may be formed between a distal end of the radial channels (220) and the perimeter of the flexible disc (232) (i.e., the sealing rings may be formed around, and may encircle, the radial channels (220)), while in other variations, one or more sealing rings may be formed between the radial channels (220). For example, in one variation, a first sealing ring may be formed between the radial channels (220) and a second, concentric sealing ring may be formed around the radial channels (220). In some embodiments, the sealing rings may have a diameter or maximum transverse dimension between about 0.02 inches (0.508 mm) and about 0.125 inches (0.318 cm) and may have a maximum thickness of between about 0.02 inches (0.508 mm) and about 0.25 inches (0.635 cm). The sealing rings may have any cross-sectional shape suitable for sealing, including but not limited to, semi-circular, triangular, square, semi-elliptical, or the like. In some variations, the seal rings may comprise a lip. In variations comprising more than one sealing ring, the sealing rings may have the same or different cross-sectional shapes.

Referring now to FIGS. 2C and 2E, the vacuum lumen (212) may fluidly couple a proximal surface of the cap (202) and the spacer lumen (222), and the flush lumen (216) may fluidly couple a proximal surface of the cap (202) and the circular channel (218). The vacuum lumen which may allow for application of negative pressure to the device and the flushing lumen may allow for application of flushing fluid to the device. The vacuum lumen (212) and the flush lumen (216) may each extend radially outward toward a surface on the proximal side (236) of the flexible disc (232) (i.e., a proximal surface). Specifically, the vacuum lumen (212) may extend from the tubular spacer lumen (222) radially outward to the proximal surface of the flexible disc (232) and the flush lumen (216) may extend from a circular channel or groove (218) formed on a distal side (238) (e.g., in a distal surface) of the flexible disc (232) radially outward to the proximal surface of the flexible disc (232). With respect to the flushing function, the circular channel (218) may be connected to and fluidly coupled with the flush lumen (216) such that flushing fluid applied to the flush port (214) may travel down the flush lumen (216) and into the circular groove (218), which, as described above, may assist transporting flushing fluid to the distal side (238) of the flexible disc (232) and to the space between the distal side (238) of the flexible disc (232) and tissue.

Referring now to FIGS. 2B and 2D, the cap (202) may comprise a plurality of radial channels (220) formed on or in the distal surface. The cap (202) may comprise any suitable number of radial channels (220), for example, two, three, four, five, or more. The radial channels (220) may extend from an external surface of the tubular spacer (204) outward toward the edge of the flexible disc. In variations in which the cap comprises first and second portions as discussed above, the radial channels (220) may extend until the edge of the first portion such that the end of the channels may be aligned with diameter of the first portion (i.e., the channels may not extend to the outer diameter of the second portion). The radial channels (220) may be formed in any suitable manner, for example, in some variations, the channels may be formed from projections or protrusions (234) extending away from (i.e., distally from) a distal side (238) of the flexible disc (232) of the cap (202) as depicted, while in other variations the radial channels (220) may be formed as grooves or indentations extending into a distal side (238) of the flexible disc (232) of the cap (202) (i.e., toward the proximal side (236)).

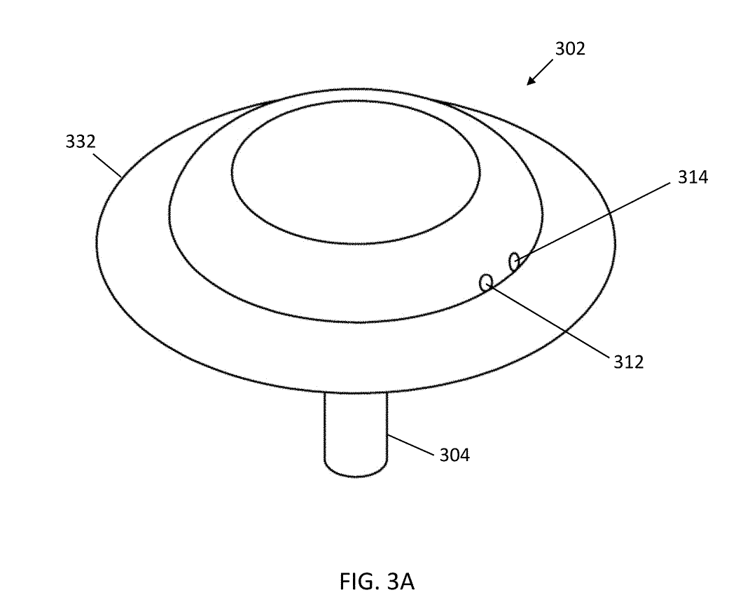

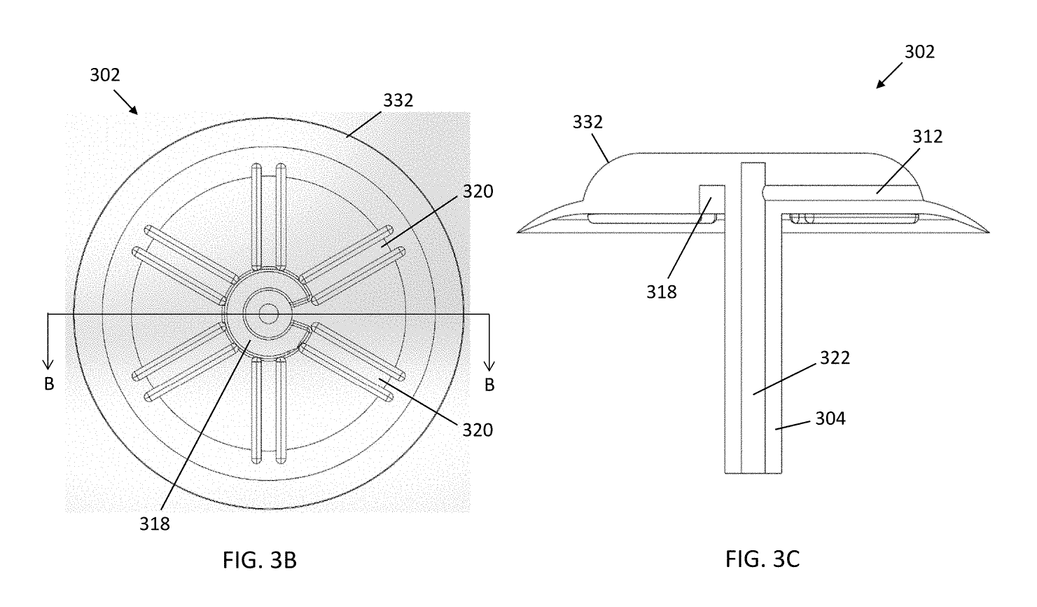

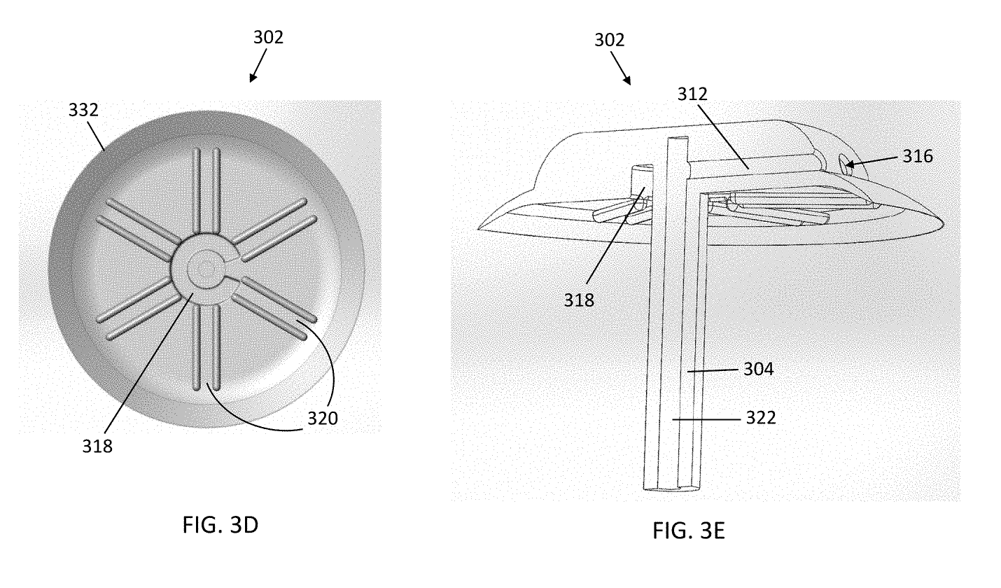

FIGS. 3A-3E depict another variation of a cap (302) that is similar to the variation depicted in FIGS. 2A-2E with like reference numbers used for like elements. Accordingly, in the variation shown in FIGS. 3A-3E, the cap (302) comprises a flexible disc (332) and a tubular spacer (304) comprising a lumen therethrough (322). The tubular spacer (304) may be integrally formed with the flexible disc (332). The cap (302) may further comprise a plurality of ports, for example, a vacuum port (310) and a flush port (314) fluidly coupled to a vacuum lumen (312) and a flush lumen (316), respectively. The vacuum lumen (312) and the flush lumen (316) may be substantially laterally aligned. The cap (302) may further comprise a circular channel or groove (318) partially circumferentially surrounding an external surface of the tubular spacer (304) to assist in flushing fluid distribution and one or more radial channels (320) to assist in negative pressure and optionally fluid distribution.

A main difference between the variations shown in FIGS. 3A-3E and FIGS. 2A-2E is that the cap (302) in FIGS. 3A-3E comprises vacuum and flush lumens aligned laterally, or in a side-by-side configuration, whereas the cap (202) in FIGS. 2A-2E comprises vacuum and flush lumens aligned vertically. Additionally, because the vacuum and flush lumens are aligned laterally, the circular groove (318) in the cap (302) is semi-circular and does not fully circumferentially surround the tubular spacer (304). In some instances it may be desirable to utilize a side-by-side configuration for the vacuum and flush lumens (as depicted in FIGS. 3A-3E) because doing so may result in a slimmer, lower-profile and/or smaller device, which may increase patient comfort.

While the vacuum and flush lumens in the variations depicted in FIGS. 2A-2E and 3A-3E are depicted next to or near one another, they need not be. In some instances, the vacuum and flush lumens may be in other locations (e.g., on opposite sides of the cap or off-set a further distance from one another (e.g., 90 degrees, 180 degrees, 270 degrees from one another)).

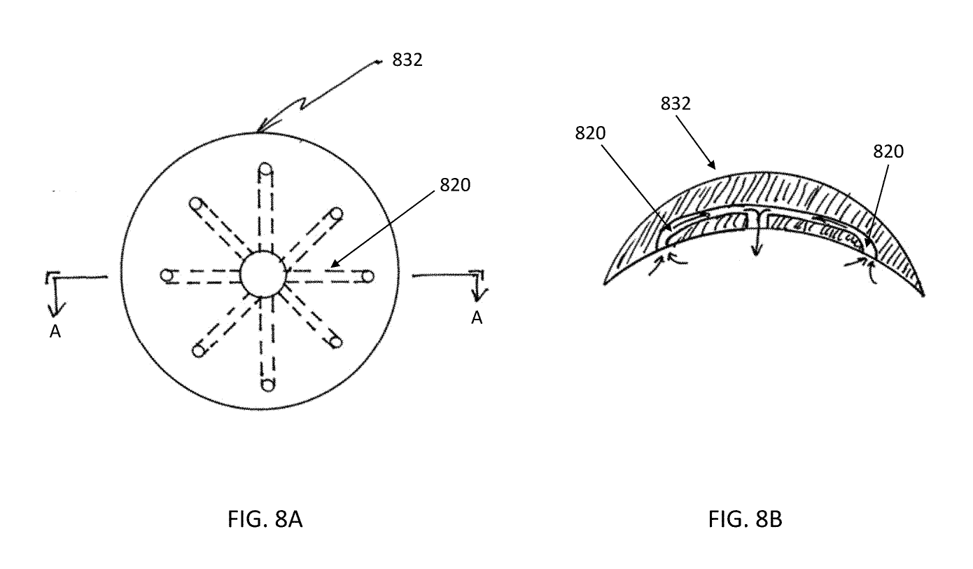

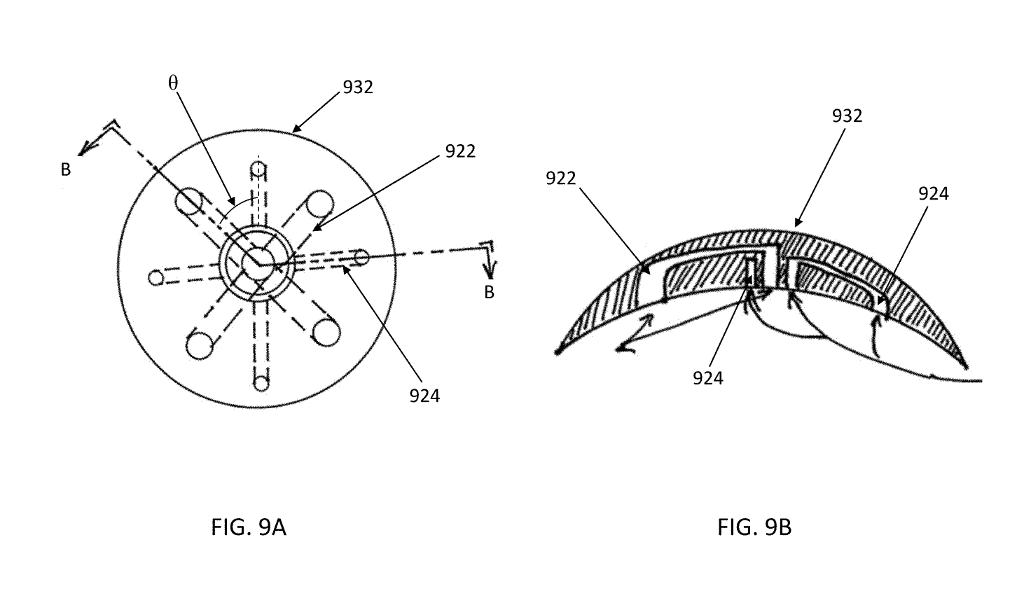

As mentioned above, while FIGS. 2A-2E and 3A-3E depict caps with radial channels formed from projections or protrusions extending away from a distal surface of the flexible disc of the cap, in some variations the channels may be formed through or integrated into the body of the flexible disc of the cap. For example, FIGS. 8A-8B and 9A-9B depict schematic variations of flexible discs (832, 932) comprising integrated channels. As shown in FIGS. 8A-8B, in some variations, the flexible disc (832) may comprise a single set of radial channels (820), which may be used to transmit negative pressure and/or flushing fluid through the flexible disc (832) toward a perimeter thereof. In these variations, the radial channels (820) may be fluidly coupled to a vacuum or flushing fluid source through a single port. In other variations, as shown in FIGS. 9A-9B, the flexible disc (932) may comprise a first set of radial channels (922) that may be used to transmit negative pressure (i.e., vacuum channels), and a second set of radial channels (924) that may be used to transmit flushing fluid (i.e., flushing channels). The channels in the first set of radial channels (922) may comprise a larger diameter (or maximum transverse dimension in variations in which the channels are non-circular), the same diameter, or a smaller diameter than the channels in the second set of radial channels (924). Additionally, while depicted with an equal number of channels in each set, the cap may comprise more channels in the first set of radial channels than in the second set of radial channels, or vice versa. Moreover, the channels in the first and second sets of radial channels may be positioned such that an angle (θ) is formed between the channels (between the central longitudinal axis of a channel in the first set of radial channels and the central longitudinal axis of a channel in the second set of radial channels). The angle (θ) may be any angle suitable for distribution of negative pressure and flushing fluid, for example, about 45 degrees or between about 30 degrees and about 60 degrees, and the angle formed between each channel in the first set of channels and each channel in the second set of channels may or may not be the same.

### First Distal Anchor

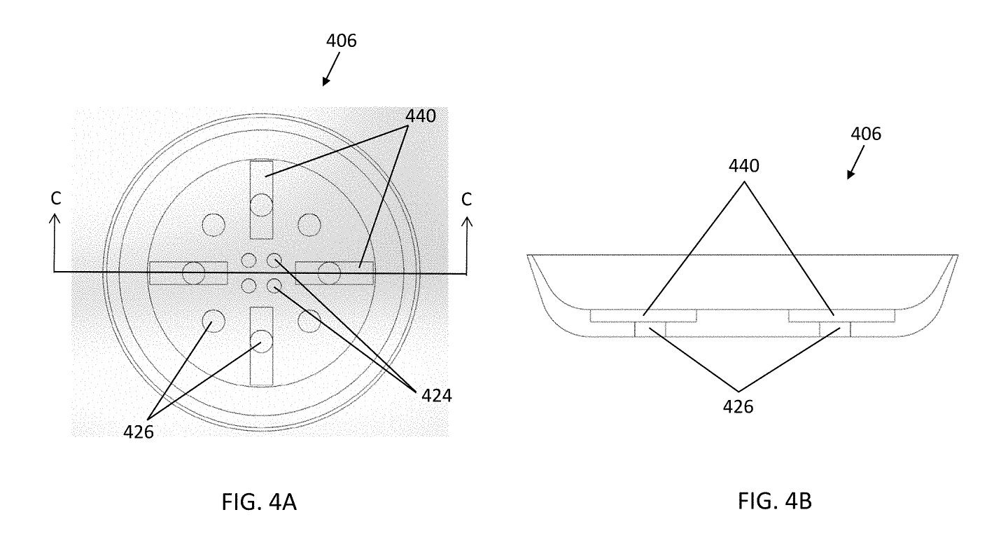

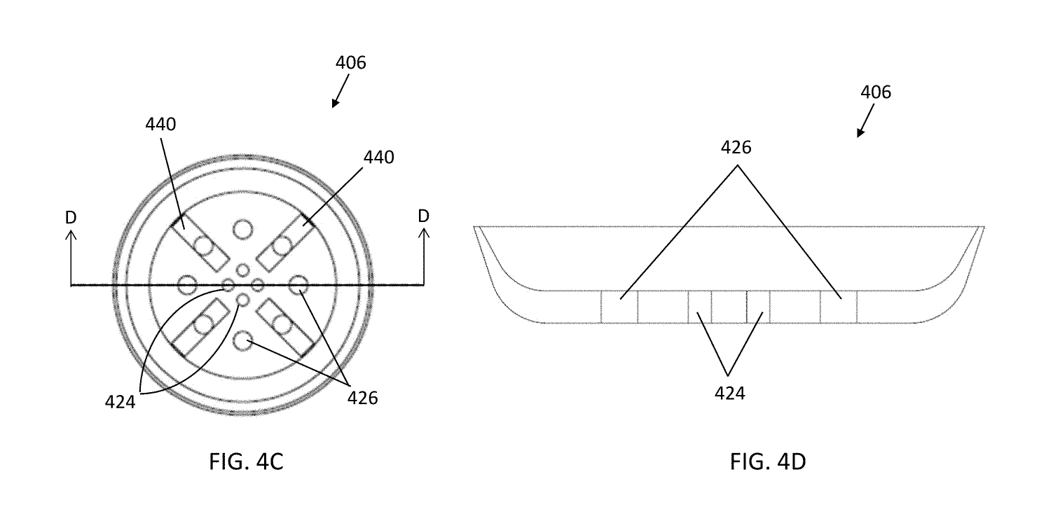

FIGS. 4A-4D depict a variation of a first distal anchor (406) comprising a plurality of openings and a plurality of channels. Specifically, FIG. 4A depicts a first top view and FIG. 4B depicts a cross-section take along line C-C in FIG. 4A. FIG. 4B depicts a second top view and FIG. 4D depicts a second cross-section taken along D-D in FIG. 4C. As shown there, the first distal anchor (406) may comprise two sets of a plurality of openings and one set of a plurality of channels.

The first set of openings may be vacuum openings (424) that may be configured to transmit negative pressure therethrough, and thus through the first distal anchor (406). The second set of openings may be flush openings (426) that may be configured to allow passage of flushing fluid therethrough, and thus through the first distal anchor (406). The vacuum openings (424) and the flush openings (426) may be arranged in concentric circles around a center point or a suture opening (not depicted) (in some variations, the suture opening may be at the center point of the first distal anchor (406)). The vacuum openings (424) and the flush openings (426) may also be arranged concentrically relative to a circumference of the first distal anchor (406).

Each of the vacuum openings (424) may comprise a first diameter and each of the flush openings (426) may comprise a second diameter. In some variations, the first diameter (i.e., the diameter of the vacuum openings (424)) may be greater than the second diameter (i.e., the diameter of the flush openings (426)), while in other variations, the first and second diameters may be equal, or the first diameter may be greater than the second diameter. While all of the vacuum openings (424) are depicted with the same diameter and all of the flush openings (426) are depicted with same diameter, this need not be the case. For example, in some variations, the distal anchor (406) may comprise a first set of vacuum or flush openings with a first diameter and a second set of vacuum or flush openings with a second, different diameter. Any diameter suitable for the transmission of negative pressure may be used for the vacuum openings (424), for example, between about 0.05 inches (0.127 cm) and about 0.40 inches (1.016 cm), while any suitable diameter for the transmission of flushing fluid may be used for the flush openings (426), for example, between about 0.01 inches (0.0254 cm) to about 0.25 inches (0.635 cm). Moreover, any suitable number of vacuum openings (424) and flush openings (426) may be included, for example, three, four, five, six, seven, eight or more each, and the number of vacuum openings (424) and flush openings (426) need not be the same.

As mentioned above, the distal anchor (406) may also comprise a plurality of channels (440) formed in or on the proximal surface thereof. Note, in this instance, the proximal surface of the first distal anchor refers to the surface facing tissue when the first distal anchor is deployed in the body. The channels (440) may be configured to transmit negative pressure radially outward to spread or distribute the negative pressure outward and increase the surface area of the first distal anchor (406) that is exposed to negative pressure. Thus, the channels (440) may assist in distributing the negative pressure and carrying it radially outward such that a seal may be formed along a circumference of the first distal anchor (406).

Any suitable number of channels may be used, for example, two, three, four, five, six, seven, eight or more, and the channels may have any dimensions suitable for transmitting the negative pressure outward without clogging. For example, in some variations, one or more channels may have a length of about 0.250 inches (6.35 mm), and/or a depth and/or width of about 0.040 inches (1.02 mm). In some variations, one or more channels may have a depth and/or width between about 0.01 inches (0.254 mm) and about 0.10 inches (2.54 mm). The channels may be positioned at any suitable location along the proximal surface, for example, at 0 degrees, 90 degrees, 180 degrees, 270 degrees, or at any angle therebetween. In some embodiments, and as depicted in FIGS. 4A-4D, the channels may be positioned 90 degrees from one another. In some instances, the channels (440) may be formed by removing material from the surface of the first distal anchor such that the channels (440) extend inward from the surface of the first distal anchor (as depicted), while in other variations, the channels may be formed by a series of projections extending outward from the surface of the distal anchor (similar to those described above with respect to the channels (220) in the cap (202)). Additionally, as depicted in FIG. 4B, one or more flush openings (426) may extend through a portion of one or more channels (440).

Additionally, in some variations, the first distal anchor (406) may further comprise one or more sealing rings formed on or from a proximal surface of the first distal anchor (406). The sealing rings may be full rings, interrupted rings, portions of rings, or form any other shape that provides a sealing or healing benefit. In some variations, the first distal anchor (406) may comprise two concentric sealing rings. In some instances, the sealing rings may be formed between a distal end of the channels (440) and the perimeter of the first distal anchor (406) (i.e., the sealing rings may be formed around, and may encircle, the channels (440)).

### Second Distal Anchor

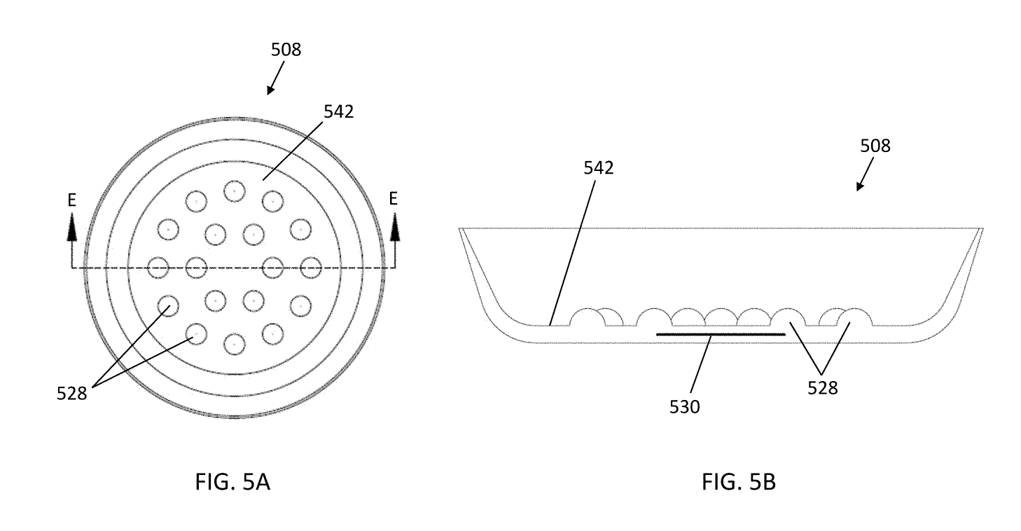

FIGS. 5A-5B illustrate a variation of a second distal anchor (508) comprising a plurality of projections (528) extending from a proximal surface (542) or side of the second distal anchor (508) and mesh (530) embedded within the second distal anchor (508). Specifically, FIG. 5A depicts a top view of second distal anchor (508) and FIG. 5B depicts a cross-sectional view taken along line E-E. In use, the projections (528) may prevent the proximal surface (542) of the second distal anchor (508) from contacting a distal surface of the first distal anchor. Thus, the projections (528) may ensure that there is a gap between the two distal anchors after tension is applied to the suture and/or negative pressure is applied to the device. The gap formed by the projections (528) contacting the distal surface of the first distal anchor may be used to transmit negative pressure to the circumference of the second distal anchor (508) so that a seal may be formed between the outer edge (at the circumference) of the second distal anchor (508) and tissue. Additionally, the gap may be used to apply fluid between the first and second distal anchors and between the outer proximal (upper) surface or edge of the second distal anchor (508) and tissue for cleaning. In some variations, the gap may be between about 0.01 inches (0.025 cm) and about 0.10 inches (0.254 cm), and preferably between about 0.035 inches (0.089 cm) and 0.05 inches (0.127 cm). The size of the gap may be selected to prevent the gap from clogging with bowel waste or sloughed off mucosal tissue.

Accordingly, any suitable number of projections (528) may be employed that maintain or create a gap sufficient for transmission of negative pressure and flushing fluid between the first and second distal anchors, for example, six, seven, eight, nine, ten, eleven, twelve, thirteen, fourteen, fifteen, sixteen, seventeen, eighteen, or more, and the projections (528) may be arranged in any suitable configuration. For example, in some variations, the projections (528) may be arranged randomly, while in other variations, the projections (528) may be arranged in concentric circles around a center point of the second distal anchor (508). For example, in some variations, the projections may be arranged in first circle with a diameter of 0.350 inches (from projection center to projection center) and a second circle with diameter of 0.700 inches (from projection center to projection center). The projections (528) may have any suitable arrangement that allows vacuum or fluid flow between them (i.e., they do not block or otherwise prevent vacuum or fluid flow).

Additionally, while depicted as dome-shaped, the projections need not be, and may have any suitable shape that maintains a gap or space between the two distal anchors (e.g., they may have a square, rectangular, triangular, trapezoidal, or the like cross-sectional shape). Moreover, while all the projections are depicted as having the same shape (i.e., as dome-shaped), this need not be the case, and one or more of the plurality of the projections (528) may have a different shape. In some variations in which the projections are dome-shaped, the diameter (or in variations in which other shaped projections are used, the maximum transverse dimension) of the projections at the proximal surface of second distal anchor may be about 0.0934 inches (2.37 mm). In some variations, the projections may have a diameter and/or a height between about 0.01 inches (0.025 cm) and 0.10 inches (0.254 cm). It may be helpful to use smaller projections (while still being large enough to allow for transmission of fluid and/or vacuum) in order to minimize the cross-sectional profile of the anchor during delivery. Moreover, in some variations, channels may be utilized instead of projections. For example, channels may be formed on the proximal side of the second distal anchor and/or on the distal side of the first distal anchor. The channels may facilitate flow radially outward and the walls of the channels may create a gap between the distal anchors.

In some variations, the second distal anchor (508) may further comprise one or more sealing rings formed on or from a proximal surface of the second distal anchor (508). The sealing rings may be full rings, interrupted rings, portions of rings, or form any other shape that provides a sealing or healing benefit. In some variations, the second distal anchor (508) may comprise two concentric sealing rings. In some instances, the sealing rings may be formed between the projections (528) and the perimeter of the second distal anchor (508) (i.e., the sealing rings may be formed around, and may encircle, the projections (528)).

As mentioned above, the second distal anchor (508) may comprise mesh (530) embedded or otherwise coupled at or near a center point of the second distal anchor (508). The mesh (530) may be configured to anchor the suture within the second distal anchor (508) and may assist in preventing the suture from tearing or ripping through the second distal anchor (508). The mesh may have any suitable size and shape, for example, it may be in the form of a rectangular or circular sheet.

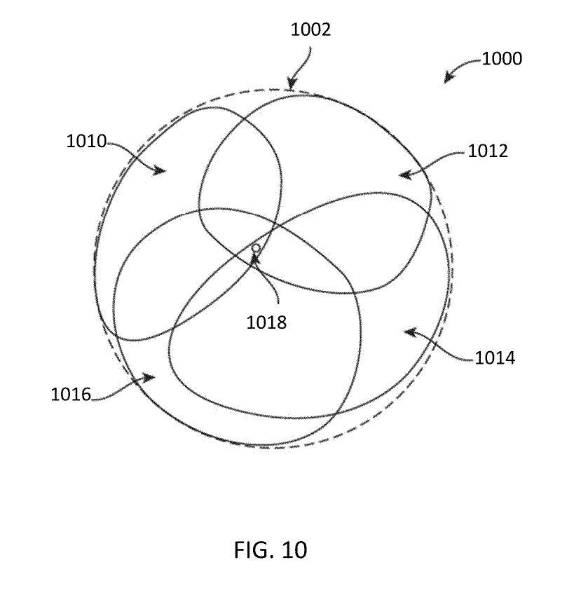

In some variations, the first and/or second distal anchor may be formed from multiple disc members or anchor portions having non-circular shapes that may be arranged to form the first and/or second distal anchor. For example, FIG. 10 depicts a variation of a distal anchor comprising one or more foldable, flexible anchor portions (1010, 1012, 1014, 1016) that are non-circular. In one embodiment, the flexible anchor portions (1010, 1012, 1014, 1016) may all be smaller than an overall circumference (1002) of the assembled distal anchor (1000), and when assembled, the distal anchor (1000) may approximate a circular shape (1002). The anchor portions (1010, 1012, 1014, 1016) may be sized and shaped to overlap one another when assembled into a circular shape in order to minimize and/or prevent leakage between the anchor portions (1010, 1012, 1014, 1016). The smaller-circumference anchor portions (1010, 1012, 1014, 1016) may be arranged in a non-concentric relationship so that each anchor portion only partially overlaps the immediately adjacent anchor portions, like in a petal configuration. The anchor portions (1010, 1012, 1014, 1016) may be held together by an attachment member (1018), such as a pin or suture. The attachment or attachment opening of each anchor portion (1010, 1012, 1014, 1016) to the attachment member 1018 may have an offset location from the center of each anchor portion (1010, 1012, 1014, 1016). The anchor portions may or may not be symmetrically arranged around the attachment member. In some variations, the anchor portions (1010, 1012, 1014, 1016) may additionally comprise interlocking or corresponding ridges and/or grooves, which may assist in aligning the flexible anchor portions (1010, 1012, 1014, 1016) relative to one another and may assist maintaining the relative positions of the anchor portions (1010, 1012, 1014, 1016). It may be advantageous to use multiple, smaller-circumference anchor portions (1010, 1012, 1014, 1016) to generate a larger circumference distal anchor (1002) because each anchor portion may be easier to fold and advance through a small diameter delivery catheter, and/or it may be easier to pass each anchor portion through the bowel and out of the body at the end of treatment as the distal anchor may come apart when released. In one embodiment, a very flexible, thinner layer (not pictured) with a circumference approximating that of circumference (1002) may be positioned above or below the multiple smaller anchor portions (1010, 1012, 1014, 1016) to facilitate assembly of the multiple portions.

In some variations, a non-circular outline may be a shape in which the perimeter is not a constant radius from a center point. Non-circular shapes include shapes with first-derivative discontinuities at one or more locations. Non-circular shapes may also be a generally circular shape with protrusions or recesses on the perimeter to accommodate a predetermined surface of a body lumen. Non-circular shapes may include, but are not limited to, ovals, ellipses, rectangles, lenses, deltoids, and bell-shapes. Additionally, non-circular shapes may include shapes in which the perimeter is a constant radius from a center point. For example, non-circular shapes may include shapes having arcs that form a circle when arranged, for example, semi-circles, quadrants, portions thereof, or the like. When non-circular, a diameter of a distal anchor portion may be understood to mean a length of the member in one dimension. For example, a line taken through a center point or a widest span of the member. In such variations, the diameters of the distal-most and inner anchor portions may be characterized as a percentage from 1% to 100% of the diameter of the proximal-most anchor portion, and may sometimes be about 5%, 10%, 20%, 25%, 30%, 35%, 40%, 45%, 50%, 55%, 60%, 65%, 70%, 75%, 80%, 85%, 90%, or 95%, or any percentage range between any two of the above percentages. In some variations, some of the anchor portions may take a shape different from one or more of the other anchor portions. For example the distal-most anchor portions may be circular, but the proximal-most anchor portions may be shaped to occlude a non-circular fistula opening. In some other variations, the distal-most anchor portions may also be non-circular in order to achieve a desired distribution of forces, for example.

### Vacuum and Flushing

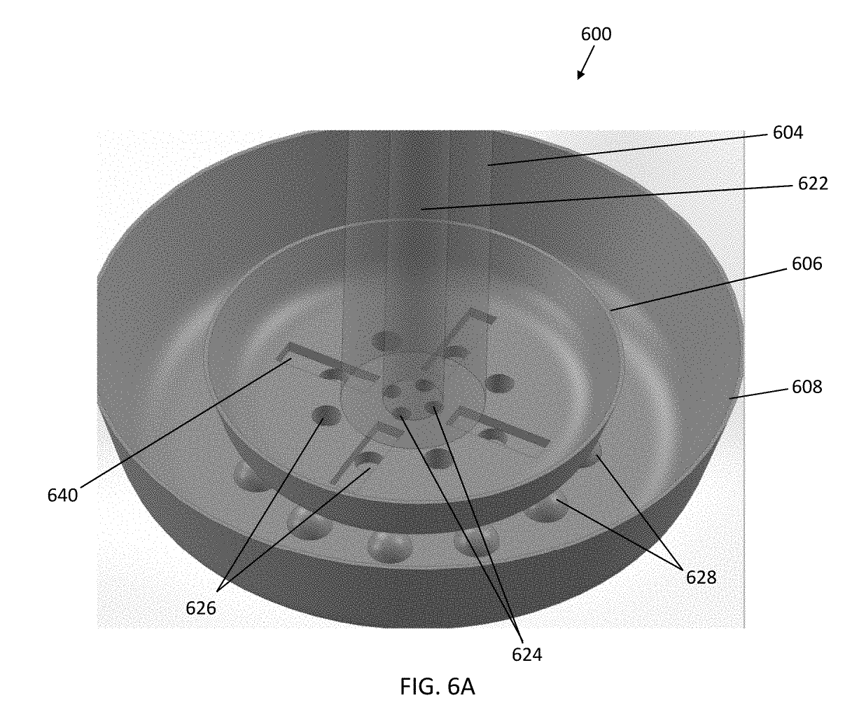

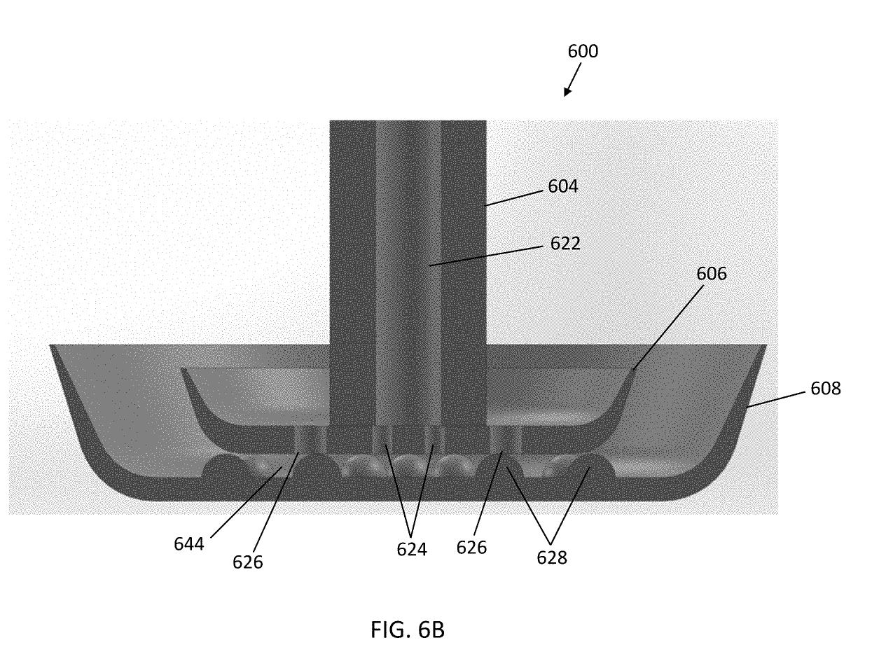

FIGS. 6A and 6B depict an angled top view and a cross-sectional view, respectively, of a distal portion of a variation of fistula treatment device (600) comprising a spacer (604), and first and second distal anchors (606, 608) in a concentrically aligned configuration (the second distal anchor (608) in FIG. 6A is not fully depicted). In this configuration, the spacer (604), and first and second distal anchors (606, 608) are concentrically aligned relative to one another, for example, using a suture disposed within the spacer lumen (622) and coupled to the center points of both the first and second distal anchors (606, 608). The suture has been removed for clarity, however, the suture can be seen in FIGS. 7C-7E. Appropriately aligning the first and second distal anchors (606, 608) relative to the spacer (604) and the cap may allow for better transmission of negative pressure and fluid through the vacuum and flushing fluid paths, respectively. For example, in this configuration, the distal end of the spacer (604) may contact a central portion of the proximal surface of the first distal anchor (606) at a location in between the concentric circles of the vacuum openings (624) and the flushing openings (626). The distal end surface of the spacer (604) may circumscribe or surround the vacuum openings (624) and the flushing openings (626) may circumscribe or surround the distal end surface of the spacer (604). This may align the spacer lumen (622) with the vacuum openings (624) such that a distal end of the spacer lumen (622) may be directly fluidly coupled to the vacuum openings (624). Additionally, in this variation, the longitudinal axes of the spacer lumen (622) and the vacuum openings (624) may be parallel. Additionally, in this configuration, an external surface of the spacer (604) may be aligned with the flushing openings (626). The distal end surface of the spacer (604) may also overlay or otherwise contact a portion (i.e., the portion closest to the center point of the second distal anchor (608)) of the one or more channels (640) in the first distal anchor (606). In some variations, the channels may extend further radially inward toward the vacuum openings (624) and may intersect and/or overlap with the spacer lumen (622).

Additionally, as shown in FIGS. 6A and 6B, the projections (628) on a proximal side or surface of the second distal anchor (608) may be in contact with a distal side of the first distal anchor (606), forming a gap or space (644) between the proximal surface of the second distal anchor (608) and the distal surface of the first distal anchor (606). This gap (644) may be fluidly coupled with both the vacuum openings (624) and the flushing openings (626) in the first distal anchor (606). While the projections (628) may overlap with or contact an area of the vacuum or flushing openings (624, 626), the projections (628) and openings (624, 626) may be configured (e.g., sized, shaped, and/or positioned) such that the projections (628) do not completely block or prevent flow through the openings (624, 626).

FIGS. 7A and 7B depict a perspective cross-sectional view and a schematic cross-sectional view of a fistula treatment device (700) similar to that described with respect to FIGS. 1A and 1B (with like elements labeled with like reference numerals) in a deployed configuration. FIG. 7C depicts the fistula treatment device (700) further comprising a suture (750) and coupled to vacuum tubing (752) comprising a Y-connector (754) and flushing tubing (756). The fistula treatment device (700) is depicted without reference to tissue in FIG. 7A, and is depicted implanted in a fistula (746) formed from a first, external surface (747) of tissue (748) to a second, internal surface (749) of tissue (748) in FIGS. 7B and 7C. The fistula treatment device (700) may comprise a cap (702), a first distal anchor (706) and a second distal anchor (708). The cap (702) may comprise a flexible disc (732) and a stiff elongate spacer or protrusion (704), a vacuum port (710) fluidly coupled to a vacuum lumen (712), a flush port (714) fluidly coupled to a flush lumen (716), a circular groove or channel (718) surrounding the spacer (704), and one or more radial channels (720). The first distal anchor (706) may comprise one or more vacuum openings (724), one or more flushing openings (726), and one or more channels (740). The second distal anchor (708) may comprise a plurality of projections (728) and mesh (730). As shown in FIG. 7C, vacuum tubing (752) may be coupled to the vacuum port (710) and flushing tubing (756) may be coupled to the flush port (714). The vacuum tubing (752) may comprise a Y-connector (754) comprising a suture seal, such that the same tubing and lumen may be used to apply negative pressure to the device and to hold the suture (750).

One or more vacuum pathways may be formed in the fistula treatment device (700) that may transmit negative pressure through the fistula treatment device to seal the first and second distal anchors to one another and to the internal tissue surface (749). For example, vacuum pathways may comprise the vacuum port (710), the vacuum lumen (712) and the spacer lumen (722) in the cap (702), the vacuum openings (724) in the first distal anchor (706), and the flushing openings (726) in the first distal anchor (706). When negative pressure is applied to the vacuum port (710), the vacuum lumen (712), the spacer lumen (722), and the vacuum openings (724) may transmit the negative pressure to a gap (744) formed between the first and second distal anchors (706, 708). As described above, the projections (728) may form or maintain the gap (744). The negative pressure may then be transmitted through the flushing openings (726) from a distal side to a proximal side (tissue facing side) of the first distal anchor (706). Thus, the cap (702) may transmit negative pressure to a portion of the proximal side of the first distal anchor (706) overlapping the spacer lumen (722), through the vacuum openings (724) to the gap (744) formed between the first and second distal anchors (706, 708), and through the flushing openings (726) to the portion of the proximal side of the first distal anchor (706) outside of the spacer lumen (722). The channels (740) may transmit the negative pressure radially outward along the proximal surface of the first distal anchor (706) to seal the first distal anchor (706) to the internal surface (749) of the tissue (748). In variations in which the channels (740) may intersect the spacer lumen (722), negative pressure may be transmitted directly from the spacer lumen (722) to the channels (740). The vacuum openings (724) may also transmit negative pressure through the first distal anchor (706) to a proximal side (tissue facing side) of the second distal anchor (708) to seal the second distal anchor (708) to the distal side of the first distal anchor (706) and to the internal surface (749) of the tissue (748). The distal surface (tissue facing surface) of the flexible disc (732) of the cap (702) may also be exposed to negative pressure. For example, negative pressure may act on the distal surface of the flexible disc (732) via a tract of the fistula (746). The radial channels (720) in the distal surface of the flexible disc (732) may carry the negative pressure radially outward to seal the flexible disc (732) of the cap (702) to the external surface (747) of the tissue (748).

FIG. 7D depicts an exemplary vacuum or negative pressure flow path with the arrows indicating the direction of flow. When the Y-connector (754) is coupled to a vacuum source (e.g., a vacuum pump) and the vacuum source is activated, negative pressure may be applied to the device (700) through the vacuum tubing (752), which may pull the first and second distal anchors (706, 708) into contact with the internal tissue surface (749). Specifically, negative pressure may be applied through the vacuum tubing (752), the vacuum port (710), and the vacuum lumen (712) in the flexible disc (732) and the spacer lumen (722), through the vacuum openings (724), and the flush openings (726) to a proximal surface of the first distal anchor (706). The negative pressure may remove the air between the proximal surface of the first distal anchor (706) and the internal tissue surface (749) and may pull the first distal anchor (706) into contact with the internal tissue surface (749). Channels on the proximal surface of the first distal anchor (706) may carry the negative pressure radially outward, such that the negative pressure is distributed across the surface of the first distal anchor (706). This may allow the negative pressure to reach the edge of the first distal anchor (706), and may create a first seal (758) around the circumference of the first distal anchor (706).

Negative pressure may also be transmitted through the vacuum openings (724) in the first distal anchor (706) to the gap (744) between the distal side of the first distal anchor (606) and the proximal side of the second distal anchor (708) to remove the air between the first and second distal anchors (706, 708). The negative pressure may be transmitted radially outward through the gap (e.g., through the spaces formed between the projections (728)), such that a seal may be formed between the first and second distal anchors (706, 708) and a second seal (760) may be formed between an edge of the second distal anchor (708) and the internal tissue surface (749) along the circumference of the second distal anchor.

Finally, negative pressure may also be transmitted through the flush openings (726) and a tract of the fistula (746) to a distal surface of the flexible disc (732) of the cap (702). The negative pressure may be carried radially outward via the radial channels (720) in the distal surface of the flexible disc (732), which may transmit the negative pressure to an outer edge of the flexible disc (732) and enable a third seal (762) to be formed between the distal surface of the flexible disc (732) of the cap (702) and the external tissue surface (747).

Thus, negative pressure may be used to hold the second distal anchor (708) to the first distal anchors (706), seal the first and second distal anchors (706, 708) to the internal tissue surface (749), and seal the flexible disc (732) of the cap (702) to the external tissue surface (747), which may anchor the device in and around the fistula and hold the device in place.

In some variations, the fistula treatment device (700) may also comprise one or more flushing pathways that may carry flushing fluid (e.g., saline or colloid, antibiotics, or a combination of like fluids) through the fistula treatment device to clean the fistula while the fistula treatment device (700) remains in place (i.e., without requiring removal of the fistula treatment device (700) from the fistula or wound). For example, flushing fluid pathways may comprise the flush port (714), the flush lumen (716), and the circular groove (718) in the cap (702), and the flushing openings (726) in the first distal anchor (706). In some variations, the flushing pathways may also comprise the spacer lumen (722). Thus, when flushing fluid is applied to the flush port (714), the flush lumen (716) may carry the flushing fluid to the circular groove (718), at which point it, the radial channels (720) may optionally carry the fluid radially outward (to further spread/distribute the flushing fluid). The tract of the fistula tract (746) (external to the spacer) may carry the flushing fluid to a proximal surface of the first distal anchor (706), and the flushing openings (726) may carry the fluid to the gap (744) formed between the first and second distal anchors (706, 708). Thus, flushing fluid may be applied through the cap (702) to the external tissue surface (747) underneath the cap (702), to the fistula tract (746) outside of the spacer lumen (722) (to a space between the spacer (704) and a wall of the tract of the fistula (746)), to the internal tissue surface (749) underneath the first distal anchor (706), to the gap (744) between the first and second distal anchors (706, 708), and to the internal tissue surface (749) underneath the second distal anchor (708). Thus, all of the surfaces of the fistula or wound may be cleaned using flushing fluid while the fistula treatment device (700) remains in-situ.

In some variations, the flushing pathway may also comprise the spacer lumen (722). In these variations, the flushing fluid may be pulled through the vacuum openings (724), into the spacer lumen (722), through the vacuum lumen (712) and out of the fistula treatment device (700). In some instances, the flushing fluid may be pulled from underneath the flexible disc (732) of the cap (702), through the tract of the fistula (746) external to the spacer (704) and then through the spacer lumen (722) such that the fistula or wound may be cleaned externally to internally. The flushing fluid from underneath the flexible disc (732) of the cap (702), and underneath both the first and second distal anchors (706, 708), may be pulled through the spacer lumen (722) instead of through the tract of the fistula (746) external to the spacer (704) (e.g., along the fistula walls). This may minimize re-contamination of the fistula tract with bowel content leakage from inside the bowel. FIG. 7E depicts an exemplary flushing fluid flow path with the arrows indicating the direction of flow.

In some variations, it may be beneficial to flush the wound or fistula while applying negative pressure. For example, applying negative pressure and flushing fluid simultaneously may assist in flushing the tissue surfaces underneath the flexible disc (732) of the cap (702), between the outer surface of the spacer (704) and the walls of the tract of the fistula (746), between the first and second distal anchors (706, 708), underneath the first distal anchor (706), and between the second distal anchor (708) and the internal surface (749) of the tissue (748). Additionally, by applying flushing fluid at a higher rate than a vacuum source can remove, it may be possible to clean or flush all of the aforementioned areas. The flushing fluid, however, may be applied at any appropriate flow rate. In some variations, applying negative pressure may also assist in removing contaminated flushing fluid from the device, the wound, and the body.

### Systems

Any of the aforementioned fistula treatment devices may be used as part of a system with other components. For example, in some variations, the fistula treatment systems may comprise any of the fistula treatment devices described here and one or more of a delivery device, a vacuum source, and a fluid flush source. In variations in which a delivery device is used, the distal anchors of the fistula treatment device may be rolled, folded, compressed, or otherwise inserted into a lumen of the delivery device for advancement through a fistula or wound to an internal tissue surface. In some variations, a rolling tool may be used to assist in rolling the distal anchors and positioning them inside the delivery device. In variations comprising a vacuum source, the system may further comprise vacuum tubing and any suitable connectors to couple the vacuum source to the vacuum port and/or vacuum lumen of the fistula treatment device. In some variations, the vacuum tubing may comprise a Y-connector comprising a suture seal. In some variations, the vacuum tubing may be formed integrally with the cap. The vacuum source may be any suitable vacuum source for the application of negative pressure, for example, the wall outlet suction provide in many hospital rooms, or an electronic or mechanical vacuum pump system used for wound drainage or negative pressure therapy, such the V.A.C.®, or PREVENA™, or the SNAP™ therapy systems by Acelity (San Antonio, Tex.) or RENASYS or PICO negative pressure wound therapy systems by Smith & Nephew (London, UK), for example. Additionally, in variations comprising a fluid source, the system may further comprise fluid tubing and any suitable connectors to couple the fluid source to the flush port and/or the flush lumen of the fistula treatment device. In some variations, the fluid tubing may be formed integrally with the cap. The fluid source may be any suitable fluid source for applying flushing fluid.

### Methods

The fistula treatment devices described herein may be used to assist in treating enteroatmospheric fistulas, short-tract fistulas, and other openings in tissue, such as wounds. In variations in which fistulas are treated, the fistulas may comprise a fistula tract formed between an external tissue surface and an internal tissue surface. It should be appreciated that in variations in which the device is used to treat enteroatmospheric fistulas, the external tissue surface may be an external skin surface and the internal tissue surface may be a surface of the bowel.

Generally, a cap of the fistula treatment device may be positioned on an external surface of tissue containing the fistula or wound, and may cover and seal the external opening of the fistula. First and second distal anchors may be positioned on an internal surface of the tissue, and may cover and seal the internal opening of the fistula or wound. A spacer may be positioned between the cap and the distal anchors, within a tract of the fistula, and may prevent both the fistula from collapsing and the cap and distal anchors from being pulled together (thereby further opening the wound) when negative pressure is applied to the device to seal it to the tissue and hold the device in place.

Methods of treating a fistula or wound may generally comprise positioning the fistula treatment device relative to the fistula. In some variations, a delivery device (e.g., a device comprising an elongate body with a lumen therethrough) may be employed to advance the first and second distal anchors through the fistula tract. For example, as mentioned above, the first and second distal anchors may be compressed or otherwise positioned within the delivery device to reduce the profile and size of the distal anchors such that they may be more easily inserted through the fistula tract to an internal opening of the fistula on the internal tissue surface. In other variations, the first and second distal anchors may be inserted through the fistula or wound without the use of a delivery device. Once the first and second distal anchors are inserted through the fistula tract, the cap may be positioned such that the spacer is disposed within the fistula tract and the flexible disc of the cap is located adjacent to the external opening of the fistula. In variations in which the spacer must be cut to the appropriate length, the length of the fistula may be measured and a portion of the spacer may be removed.

Once the first and second distal anchors are positioned on the internal surface of the tissue, the spacer is positioned within the fistula tract, and the flexible disc of the cap is positioned on an external tissue surface, tension may be applied to a proximal end of the suture to align and further position the first and second distal anchors and the cap (including the flexible disc and the spacer). Applying tension to the suture may concentrically align the first and second distal anchors, the spacer, and the flexible disc of the cap. In some variations, applying tension to the suture may also position the first distal anchor against the internal tissue surface and the second distal anchor partially against the first distal anchor (i.e., at a central portion of the second distal anchor) and partially against the internal tissue surface (i.e., around the edge of second distal anchor). For example, the projections on the proximal side or surface of the second distal anchor may be pulled or otherwise placed into contact with a distal surface of the first distal anchor, which as described above, may form or maintain a gap or space between the first and second distal anchors. In other variations, applying tension to the suture may concentrically align the first and second distal anchors and the cap, but the distal anchors may not contact tissue and/or the second distal anchor may not contact the first distal anchor until negative pressure is applied (i.e., a space may remain between the proximal surface of the projections and the distal surface of the first distal anchor). It should be appreciated that in variations in which the suture is disposed in the vacuum lumen of the cap, the suture may remain within the vacuum lumen while negative pressure is applied through the vacuum lumen.

Once the cap (including the flexible disc and spacer) and the first and second distal anchors are appropriately aligned, negative pressure may be applied to the fistula treatment device. In some variations, applying negative pressure may position the first distal anchor against the internal tissue surface, and the second distal anchor against the first distal anchor and against the internal tissue surface. In other variations, the first distal anchor and second distal anchor may already be positioned against the internal tissue surface and the surface of the first distal anchor and internal tissue surface, respectively, prior to applying negative pressure. Either way, the application of negative pressure to the fistula treatment device may seal the first and second distal anchors to an internal surface of the tissue and the cap to an external surface of the tissue and may hold the device in place.

Specifically, the vacuum port and/or vacuum lumen may be coupled with a vacuum source (for example, via vacuum tubing). Accordingly, applying negative pressure may comprise activating the vacuum source and applying negative pressure through the vacuum port, the vacuum lumen, the spacer lumen, and the vacuum openings to the gap between the first and second distal anchors, and the flushing openings. In variations in which the second distal anchor is already stacked with or abutting against (e.g., through the contact of the proximal surface of the protrusions with the distal surface of the first distal anchor) the first distal anchor, applying negative pressure may comprise applying negative pressure through the vacuum openings to the gap formed by the protrusions between the first and second distal anchors. Additionally, applying negative pressure may further comprise distributing or carrying the negative pressure radially outward through channels formed in the proximal surface of first distal anchor and through the space formed between the protrusions on the proximal surface of the second distal anchor. Distributing the negative pressure in this way may enable the negative pressure to act on the edges of the first and second distal anchors, thereby forming two seals, a first seal along the circumference of the first distal anchor and a second seal along the circumference of the second distal anchor. In some instances, the negative pressure may pull the tissue disposed between the outer edges of the first and second distal anchors such that a lip or bump is formed, which may further assist in holding the device in place.

Additionally, applying negative pressure may also comprise applying negative pressure through the fistula tract external to the spacer (i.e., between an external surface of the spacer and the fistula walls), to a distal surface of the flexible disc of the cap. The negative pressure may be carried radially outward by channels formed on a distal side or surface of the flexible disc, which may allow the negative pressure to reach the edge of the flexible disc, act on a larger surface area of the flexible disc, and form a seal around the circumference of the flexible disc of the cap.

In some variations of the methods described here, the fistula or wound may be cleaned without removing the fistula treatment device, for example, by applying flushing fluid to the fistula. In these variations, the method may generally comprise applying negative pressure to the fistula treatment device to anchor the device on the internal and external sides of the tissue as described above, and flushing the tract of the fistula using a flushing fluid with the fistula treatment device in-situ. In some variations, the method may further comprise inserting the first and second distal anchors through the fistula (with or without a delivery device) and positioning the cap and the first and second distal anchors as described above.

Flushing a tract of the fistula may comprise coupling a fluid source to the flush port and/or flush lumen in the cap of the fistula treatment device and applying flushing fluid to the fistula through the device. For example, applying the flushing fluid to the device may comprise inserting flushing fluid through the flush port and/or the flush lumen to the circular or semi-circular groove surrounding the spacer such that it travels down the fistula tract outside of the spacer (e.g., between the spacer and the fistula wall), through the flushing openings in the first distal anchor, to the gap formed between the first and second distal anchors, and optionally through the spacer lumen. In some variations, applying the flushing fluid may also comprise carrying the flushing fluid radially outward using the channels formed in the distal surface of the flexible disc of the cap, the channels formed in the proximal surface of the first distal anchor, and/or the gap formed between the protrusions on the proximal surface of the second distal anchor. Utilizing these features to carry the flushing fluid radially outward may assist in distributing the flushing fluid, which may result in a better cleaning of the fistula or wound. Thus, applying flushing fluid to the fistula through the device may comprise flushing the external surface of the tissue underneath the flexible disc of the cap, flushing the fistula tract, flushing the internal surface of the tissue underneath the first and second distal anchors, and flushing the gap formed between the first and second distal anchors.

Negative pressure and flushing fluid may be applied simultaneously. In some variations, the method may comprise flushing the fistula a plurality of times (i.e., intermittently applying flushing fluid). In other variations, the method may comprise flushing the fistula a single time. Moreover, the method may comprise removing contaminated flushing fluid through the spacer lumen, flushing contaminated flushing fluid into the bowel, or a combination of two. In variations in which the device may comprise a plurality of spacers with different (e.g., decreasing) diameters, the method may further comprise removing the cap, replacing the previously deployed spacer with a new smaller spacer, repositioning the cap (including the new spacer), and reapplying negative pressure to re-seal the fistula treatment device. Additionally, in some variations, the method may further comprise removing the fistula treatment device from a patient's body.

Although the foregoing implementations has, for the purposes of clarity and understanding, been described in some detail by of illustration and example, it will be apparent that certain changes and modifications may be practiced, and are intended to fall within the scope of the appended claims. Additionally, it should be understood that the components and characteristics of the devices described herein may be used in any combination, and the methods described herein may comprise all or a portion of the elements described herein. The description of certain elements or characteristics with respect to a specific figure are not intended to be limiting or nor should they be interpreted to suggest that the element cannot be used in combination with any of the other described elements.

1. (canceled)

2: A fistula treatment device comprising:
a cap comprising a vacuum lumen;
a rigid tubular spacer coupled to and extending from a distal surface of the cap, wherein the spacer comprises a spacer lumen therethrough, and wherein the spacer lumen is fluidly coupled to the vacuum lumen;
a first distal anchor operably connected to the cap and the tubular spacer, wherein the first distal anchor comprises a plurality of openings therethrough, and wherein the plurality of openings are fluidly coupled to the vacuum lumen through the spacer lumen; and
a second distal anchor operably connected to the cap, the tubular spacer, and the first distal anchor,
wherein the vacuum lumen, the spacer lumen, and the plurality of openings form vacuum pathways that transmit negative pressure through the device to seal the first and second distal anchors to each other and to a tissue surface.

3: The device of claim 2, wherein the cap and the tubular spacer are integrally formed.

4: The device of claim 2, wherein the tubular spacer is threaded on a proximal end.

5: The device of claim 4, wherein a length of the tubular spacer is adjustable.

6: The device of claim 2, wherein the tubular spacer is bio-absorbable.

7: The device of claim 2, wherein the vacuum lumen extends from the spacer lumen radially outward to a proximal surface of the cap.

8: The device of claim 2, wherein a distal surface of the cap further comprises a circular channel formed around the tubular spacer.

9: The device of claim 8, wherein the cap further comprises a flush lumen fluidly coupling a proximal surface of the cap and the circular channel.

10: The device of claim 9, wherein the flush lumen extends radially outward from the circular channel to the proximal surface of the cap.

11: The device of claim 10, wherein the flush lumen and the vacuum lumen are side-by-side.

12: The device of claim 10, wherein the vacuum lumen and the flush lumen are vertically aligned, and wherein the vacuum lumen is disposed proximally of the flush lumen.

13: The device of claim 2, wherein a distal surface of the cap comprises radial channels or grooves.

14: The device of claim 2, wherein at least one of the cap, the tubular spacer, the first distal anchor, and the second distal anchor is radiopaque.

15. (canceled)

16: The device of claim 2, wherein the cap, the first distal anchor, and the second distal anchor are flexible.

17-21. (canceled)

22: The device of claim 2, wherein the first distal anchor further comprises a central suture opening.

23: The device of claim 22, wherein the second distal anchor comprises embedded mesh.

24: The device of claim 22 further comprising a suture, wherein the suture is threaded through the vacuum lumen, the spacer lumen, and the suture opening, and is coupled to the mesh.

25: The device of claim 2, wherein a proximal surface of the second distal anchor comprises one or more projections.

26-69. (canceled)
